(19) United States
(12) Patent Application Publication
(10) Pub. No.: US 2020/0163843 A1
(43) Pub. Date: May 28, 2020

(54) SYSTEM AND METHOD FOR MONITORING ADMINISTRATION OF NUTRITION

(71) Applicant: Nathania Alexandra Francis, Nesconset, NY (US)

(72) Inventor: Nathania Alexandra Francis, Nesconset, NY (US)

(21) Appl. No.: 16/777,170

(22) Filed: Jan. 30, 2020

Related U.S. Application Data
(63) Continuation of application No. 14/888,342, filed on Oct. 30, 2015, now abandoned.
(60) Provisional application No. 61/818,168, filed on May 1, 2013.
(60) Provisional application No. 61/928,835, filed on Jan. 17, 2014.

(51) Int. Cl. A61J 15/00; A61B 5/00; G16H 10/60; G16H 40/63

(52) U.S. Cl. CPC A61J 15/0084; A61B 5/4866; A61J 15/0088; A61B 2560/0475; G16H 10/60; G16H 40/63; A61J 15/0076

(57) ABSTRACT

A system for monitoring a patient's nutrition administration, the system including a processing device and a memory storing instructions executable by the processing device for: accessing nutrition administration information for a plurality of patients describing nutrition administered by different forms to the plurality of patients; accessing medical information about the plurality of patients from a healthcare information system; accessing nutritional requirement data describing a nutritional requirement for the plurality of patients; determining a difference between a nutritional requirement for a patient of the plurality of patients based on the nutritional requirement data, an amount of a nutrient administered to the patient, and the nutrition administration information; and integrating the nutrition administration information for the patient, the medical information for the patient, and the nutritional requirement data for the patient, for determining a nutritional adjustment for administering the amount of the nutrient to the patient based on the difference.

This application is a continuation of U.S. application Ser. No. 14/888,342 filed on Oct. 30, 2015, which claims the benefit of priority to International Application No. PCT/US2014/036404 filed on May 1, 2014, which claim the benefit of priority to U.S. Provisional Application No. 61/818,168 filed on May 1, 2013, and U.S. Provisional Application No. 61/928,835, filed on Jan. 17, 2014, the contents of which are herein incorporated by reference in their entirety.

### FIELD OF THE INVENTION

The present invention generally relates to a system and method for monitoring administration of nutrition. More particularly, the invention relates to a system and method for monitoring administration of nutritional formula to a patient, and determining an accrued deficit or surplus of a nutrient compared to the patient's nutritional requirements.

### BACKGROUND

Patients that are unable to swallow or take food or medications by mouth e.g., patients on ventilator support, critical illness, trauma, sedated, or with dysphagia after stroke, typically receive nutrition intravenously through a catheter, which is referred to as parenteral nutrition, or through a feeding tube, which is referred to as enteral nutrition. Enteral feeding is typically used to feed patients when there is no contraindication of the gastrointestinal tract present. In critical care areas, and for long term use, enteral feeding is advantageous and the preferred method of feeding, because it provides adequate nutrition and preserves the intestinal mucous membrane.

While the administration of enteral nutrition is effective and beneficial for patients, the effectiveness can depend on proper ordering, administration, and monitoring of the enteral feeding. The administration of enteral nutrition is a multidisciplinary process that can be hindered due to breaks in communication, complexities of modern healthcare, and decreased staff, both at the bedside and at the nutritional support level. Improper administration of enteral food has been associated with complications, such as delayed wound healing and development of pressure ulcers.

Administration of enteral nutrition can be implemented using enteral feeding pumps that generally deliver a specified volume of enteral formula to the patient at a rate selected by the user. For example, a healthcare provider can set an enteral feeding pump to administer a prescribed volume of formula at a prescribed rate. The pump measures and controls the rate of flow to deliver the formula at the set rate. However, if there is a slight error in the rate of flow measurement, the error can propagate over time and cause a deficit in the amount of formula actually delivered to the patient.

Despite the attention given by healthcare providers to the management and practice of providing adequate nutrition to patients, the patients may be undernourished. There are a number of common scenarios in which the prescribed delivery of enteral nutrition can be impeded or interrupted. For example, a pump can malfunction, tubing through which the formula is delivered can become kinked or clogged preventing a flow of formula to the patient, or doctor's orders may be prescribed to discontinue feeding, such as before or after a medical procedure. If these impedances and interruptions are not detected or compensated for, they can cause underfeeding of patients.

Historically it is a known fact that there is a direct correlation between malnutrition and adverse outcomes, which has been well documented since the 1930's. The body's natural metabolic response to illness leads to wasting of the lean body mass, which then further impairs immune function in the body. Studies have shown detrimental clinical outcomes in patients that are critically ill and are underfed and malnourished. These patients are at a much higher risk of infection, developing pressure ulcers, delay in wound healing, prolonged hospital stays, morbidity, mortality, and an overall increase in their cost of care.

Owing to the relationship between inadequate provision of nutrition to enterally fed patients and pressure ulcers, there is a trend for administrative or insurance bodies to require greater oversight of the provision of nutrition of enterally fed patients that have developed pressure ulcers. U.S. federal and regulatory agencies have been advocating for pressure ulcer prevention strategies. In October 2008, the Centers of Medicare and Medicaid Services identified pressure ulcers stages III and IV as “never events,” concluding that such pressure ulcers were preventable and the cost associated in treatment will not be reimbursed. A trend continues of pay for performance, where the governmental or private insurance payers will withhold reimbursement for medical care related to pressure ulcers when such ulcers develop in critically ill patients, unless the care provider can show proof of proper provision of nutrition to the patient, along with proper documentation of performance of preventive pressure ulcer intervention measures. Accordingly, there is a need for care providers to maintain accurate and well documented records of proper administration of a nutrition therapy and regimen, especially in the enterally fed patient population.

### SUMMARY

The present disclosure provides a nutrition monitoring system for monitoring a patient's nutrition administration. The system includes a processing device and a memory to store instructions that, when executed by the processing device, cause the processing device to perform operations. The operations include accessing nutrition administration information for a plurality of patients describing nutrition administered by different forms to the plurality of patients, accessing medical information about the plurality of patients from a healthcare information system, and accessing nutritional requirement data describing at least one nutritional requirement for the plurality of patients. The operations further include determining a difference between at least one nutritional requirement for a patient of the plurality of patients based on the nutritional requirement data, an amount of a nutrient administered to the patient, and the nutrition administration information. The operations also include integrating the nutrition administration information for the patient, the medical information for the patient, and the nutritional requirement data for the patient, for determining a nutritional adjustment for administering the amount of the nutrient to the patient based on the difference.

In an embodiment, the medical information includes at least one of a lab result, a diagnostic testing result, a medical diagnosis, a medical condition, and a wound monitoring result for the plurality of patients.

In an embodiment, the operations further include storing nutrition administration information for nutrition administered to the patient over time, storing the patient's nutritional requirements over time, and determining over a selectable accumulation time period an accumulation of the difference for the nutrient. Additionally, determining the nutritional adjustment is further based on a percentage of the accumulation of the difference specified by a selectable compensation percentage, wherein the compensation percentage is at least one of received by user input and determined using the medical information.

In an embodiment, the nutritional adjustment is determined for a selectable compensation time period having a plurality of intervals that each include a plurality of sub-intervals, and for a selectable portion of the sub-intervals for each interval. Additionally, the second time period and the portion of the sub-intervals are at least one of received by user input and determined based on the medical information.

In an embodiment, the different forms of nutrition administered to the plurality of patients include at least two of enteral formula, parenteral formula, and orally administered nutrition. Additionally, accessing nutrition administration information for the plurality of patients includes receiving measurement data captured from at least one enteral feeding pump or parenteral infusion pump that administers nutrition.

In another embodiment, the operations further include generating control signals to control a feeding pump or infusion pump to administer nutrition to the patient based on the adjusted amount of nutrition to be administered to the patient.

In another embodiment, the operations further include monitoring nutrition status related data associated with the medical information of a patient of the plurality of patients that indicates a change in, or a risk factor associated with, a nutritional requirement of the patient, the nutrition status data related to at least one of development of a skin wound, condition of a skin wound, diagnostic result data, and a medical diagnosis. Additionally, the operations further include generating an indicator when the nutrition status related data indicates at least one of: the patient was admitted to a health care facility with a skin wound, the patient developed a skin wound, the skin wound has increased in a dimension or worsened in condition, a new medical condition has developed, a medical condition has worsened, recording the indicator and a corresponding timestamp, and receiving a nutritional intervention recommended by a medical practitioner.

In still another embodiment, the nutritional adjustment is further determined based on the nutrition status related data. Additionally, generating the indicator includes generating a recommendation for determining the nutritional adjustment.

In a further embodiment, the operations further include monitoring nutrition status related data associated with the medical information of a patient of the plurality of patients that indicates a change in, or a risk factor associated with, a nutritional requirement of the patient, the nutrition status data related to at least one of development of a skin wound, condition of a skin wound, diagnostic result data, and a medical diagnosis. Additionally, the operations include selecting, based on the nutrition status related data, at least one of an enteral formula from a plurality of enteral formulas to administer to the patient, and an administration parameter for administering the selected enteral formula to the patient, the administration parameters including at least one of rate of formula administration, formula volume to be administered, and time duration of formula administration.

In still another embodiment, the operations further include receiving a user input designating one formula of a plurality of formulas as a reference formula, comparing another formula of the plurality of formulas to the reference formula, and determining a difference in nutrition content for at least one nutrient between the other formula and the selected formula. In this embodiment, the nutritional adjustment is determined for the reference formula and for the other formula.

In another embodiment, accessing nutrition administration information for the plurality of patients includes receiving a weight measurement of a reservoir of formula. In this embodiment, the operations further comprise determining a weight of formula that was actually output by a pump administering the formula from the reservoir based on a change in the received weight measurement.

In still another embodiment, the operations further include receiving expected pump output data describing an amount of formula that the pump is expected to output, determining a difference between a weight of the amount of formula described by the expected feeding pump output data and the determined weight, and generating control signals to control the feeding pump to adjust the weight of the formula output by the feeding pump based on the determined difference.

In an additional embodiment, the operations further include determining whether the weight measurement indicates the occurrence of an event related to movement of the bag or patient.

In a further embodiment, the nutrition administration describes nutrition of at least two different forms of nutrition administered to the patient, the two different forms selected from enteral formula, parenteral formula, and oral nutrition.

In another embodiment, accessing the nutrition information for a patient of the plurality of patients includes simulating administration of enteral formula by a pump.

In another embodiment, the pump operates using gravity to administer the formula.

In another embodiment, the pump is remote from the processing device.

In another aspect of the disclosure, a processing system is provided for monitoring a patient's enteral feeding. The processing system includes a processing device and a memory to store instructions that, when executed by the processing device, cause the processing device to perform operations. The operations include providing a graphical user interface (GUI) configured for a user to enter and update a plurality of patient status parameters related to at least one nutritional requirement, and select at least one nutritional formula from a plurality of nutritional formulas. The operations further include receiving initialization information to initialize at least a portion of the plurality of patient status parameters and the selected at least one nutritional formula, receiving, via the GUI, patient status update information each time a user updates the plurality of patient status parameter, and receiving, via the GUI, nutritional formula selection information each time a user updates the selected at least one nutritional formula.

Additionally, the operations include accessing information describing nutritional content of the plurality of nutritional formulas and calculating a change in the at least one nutritional requirement of the patient based on received patient status update information. The operations further include indicating a nutritional formula of the plurality of nutritional formulas that can satisfy the patient's at least one nutritional requirement based on the change in the patient's at least one nutritional requirement and the nutritional content of the plurality of nutritional formulas, and calculating a difference between the patient's at least one nutritional requirement and corresponding nutritional content that can be provided by the selected or indicated nutritional formula over a selectable time interval based on received nutritional formula selection information and the plurality of patient status parameters.

In a further embodiment, selecting the nutritional formula is further based on at least one of ingredients, cost, and availability of the plurality of nutritional formulas.

In another embodiment, the operations further include calculating, based on the change in the patient's at least one nutritional requirement and the nutritional content of the plurality of nutritional formulas, formula administration parameters for administering the selected or indicated nutritional formula, and calculating, based on received formula selection information, the nutritional content of the selected or indicated at least one nutritional formula, and the plurality of patient status parameters, formula administration parameters for administering the selected or indicated nutritional formula. The formula administration parameters include at least one of rate of formula administration, formula volume to be administered, and time duration of formula administration.

In a further embodiment, the plurality of patient status parameters includes any combination of parameters selected from the group of parameters consisting of the patient's health status, prescribed medication, prescribed medical intervention, weight, diagnostic result, activity level, feedback, allergies, history of administered formula, caregiving level available to the patient, patient's place of residence, patient financial means, patient insurance coverage, and patient preference of nutritional formula.

In another embodiment, calculating the difference further includes consulting aggregated data for a plurality of patients, and basing the calculation on the aggregated data.

In still another embodiment, at least a portion of the initialization information, patient status update information, and nutritional formula selection information is received from a processor of a digital information system storing data related to the patient, the digital information system including at least one of an electronic medical record for the patient, a hospital information system, a laboratory information system, and an allergy information system.

In another embodiment, the operations further include determining compatibility of each selected or indicated nutritional formula with the patient. In this embodiment, the compatibility describes a characteristic of the associated selected or indicated nutritional formula as it relates to the plurality of patient status parameters. Additionally, the characteristic of the associated selected or indicated nutritional formula includes at least one of nutritional content, allergens, administration requirements, nutritional requirement, storage requirements, ingredients, cost, and availability of the selected or indicated nutritional formula.

In another embodiment, the operations further include receiving pump output data indicative of an amount of nutritional formula output by an enteral feeding pump for administration to the patient, determining a difference between the at least one nutritional requirement of the patient and nutritional content provided by the amount of nutritional formula output by the feeding pump as indicated by the pump output data, and determining an adjusted amount of nutritional formula to be output by the feeding pump based on the determined difference.

In a further embodiment, determining the adjusted amount of nutritional formula includes configuring the adjusted amount for one of: a continuous administration for a selectable duration of time at a selectable rate, and multiple discontinuous administrations for respective selectable durations of time at respective selectable rates with selectable durations of intervening time intervals.

In an additional embodiment, the operations further include storing a history of the received pump output data, storing determined differences between the at least one nutritional requirement of the patient and nutritional content provided by the amount of nutritional formula output by the feeding pump, wherein the determined difference is determined for a selectable interval of time. The operations further include storing received pump output data related to administration of the adjusted amount.

In a further aspect of the disclosure, a system is provided for measuring output of enteral formula output by an enteral feeding pump. The system includes a measuring device that measures a weight of a reservoir of enteral formula from which the feeding pump pumps the formula, wherein the measuring device outputs weight data based on the measured weight, a processing device, and a memory to store instructions that, when executed by the processing device cause the processing device to perform operations. The operations include receiving the weight data and determining a weight of formula that was actually output by the feeding pump based on the weight data.

In an additional embodiment, the operations further include accessing nutritional requirement data describing at least one nutritional requirement for a patient to whom the formula output by the feeding pump is administered, determining a difference between the at least one nutritional requirement and corresponding nutritional content provided by an amount of the formula output by the feeding pump as indicated by the determined weight, and determining an adjusted weight of formula to be output by the feeding pump based on the determined difference.

In another embodiment, the operations further include receiving expected feeding pump output data describing an amount of formula that the feeding pump is expected to output, determining a difference between a weight of the amount of formula described by the expected feeding pump output data and the determined weight, generating control signals to control the feeding pump to adjust the weight of the formula output by the feeding pump based on the determined difference, and determining if the weight data indicates the occurrence of an event related to movement of the bag or patient.

In a further aspect of the disclosure, a method for monitoring administration of nutrition to a patient is provided. The method includes accessing nutrition administration information for a plurality of patients describing nutrition administered to the plurality of patients of at least two forms of nutrition, selected from parenteral formula, enteral formula, and orally administered nutrition, accessing medical information about the plurality of patients from a healthcare information system, and accessing nutritional requirement data describing at least one nutritional requirement for the plurality of patients. Additionally, the method includes determining a difference between at least one nutritional requirement for a patient of the plurality of patients based on the nutritional requirement data and an amount of a nutrient administered to the patient based on the nutrition administration information and integrating the nutrition administration information for the patient, the medical information for the patient, and the nutritional requirement data for the patient for determining a nutritional adjustment for administering the amount of the nutrient to the patient based on the difference.

Any combination of the above features is envisaged. Other objects and features will become apparent from the following detailed description considered in conjunction with the accompanying drawings, wherein like reference numerals in the various drawings are utilized to designate like components. It is to be understood, however, that the drawings are designed as an illustration only and not as a definition of the limits of the invention.

### BRIEF DESCRIPTION OF THE DRAWINGS

FIG. 1A shows a block diagram of an exemplary nutritional monitor system in accordance with the present disclosure;

FIG. 1B is a schematic drawing of a nutritional monitoring system operably connected to an enteral pump.

FIG. 2 shows an exemplary screen shot of a graphical user interface, showing a patient's nutritional requirements, generated by a processing device of the nutritional monitor system;

FIG. 3A shows an exemplary screen shot of a graphical user interface, showing a graph of daily deficits and surpluses of nutritional administration over a time period, generated by a processing device of the nutritional monitor system;

FIG. 3B shows an exemplary screen shot of a graphical user interface, showing a graph of daily nutrition administration over a time period, generated by a processing device of the nutritional monitor system;

FIG. 3C shows an exemplary screen shot of a graphical user interface, showing a graph of daily activity related to nutrition administration over a time period, generated by a processing device of the nutritional monitor system;

FIG. 3D shows an exemplary screen shot of a graphical user interface, showing a graph of daily accumulated deficits and surpluses of nutrition administration over a time period, generated by a processing device of the nutritional monitor system;

FIG. 4 shows an exemplary screen shot of a graphical user interface, showing diagnostic results, generated by a processing device of the nutritional monitor system;

FIG. 5 shows an exemplary screen shot of a graphical user interface, showing formula comparisons, generated by a processing device of the nutritional monitor system;

FIG. 6A shows an exemplary screen shot of a graphical user interface, showing nutritional consultation information, generated by a processing device of the nutritional monitor system;

FIG. 6B shows an exemplary screen shot of a graphical user interface, showing wound condition monitoring information, generated by a processing device of the nutritional monitor system;

FIG. 7 shows an exemplary screen shot of a graphical user interface, showing nutritional recommendation calculation information, generated by a processing device of the nutritional monitor system;

FIG. 8 shows an exemplary screen shot of a graphical user interface, showing current continuous enteral feeding administration information and nutritional history information, generated by a processing device of the nutritional monitor system;

FIG. 9 shows an exemplary screen shot of a graphical user interface, showing current bolus enteral feeding administration information and nutritional history information, generated by a processing device of the nutritional monitor system;

FIG. 10 shows an exemplary screen shot of a graphical user interface, showing current oral nutrition administration information and nutritional history information, generated by a processing device of the nutritional monitor system;

FIG. 11 shows an exemplary flowchart of steps performed by the monitor system, in accordance with the present disclosure; and

FIG. 12 shows an exemplary embodiment of an enteral web server of the nutritional monitor system shown in FIG. 1.

### DETAILED DESCRIPTION

The following sections describe exemplary embodiments of the present disclosure. It should be apparent to those skilled in the art that the described embodiments of the present disclosure provided herein are illustrative only and not limiting, having been presented by way of example only. All features disclosed in this description may be replaced by alternative features serving the same or similar purpose, unless expressly stated otherwise. Therefore, numerous other embodiments of the modifications thereof are contemplated as falling within the scope of the present disclosure as defined herein and equivalents thereto.

Exemplary embodiments include a system and method for monitoring and documenting nutrition administration to ensure proper nourishment of patients who are fed enterally, parenterally, or orally. An amount of formula output by a pump is determined, which is used to determine nutritional input to a patient. Nutritional input to the patient and patient nutritional requirements are compared. A system and method for determining a delivery parameter for delivering an enteral formula to a patient is described in U.S. Pat. No. 8,021,322, the contents of which are incorporated by reference herein in their entirety.

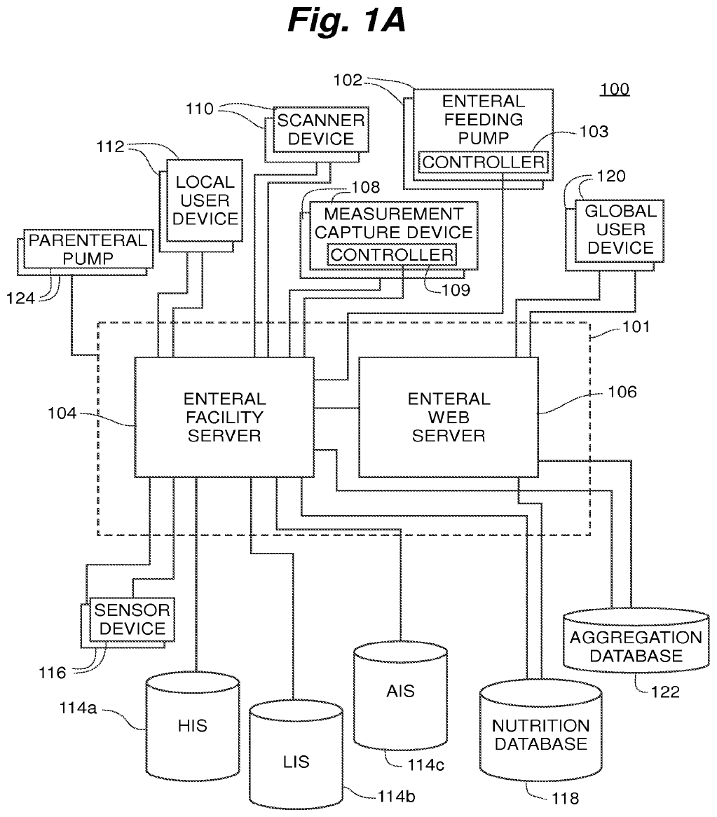

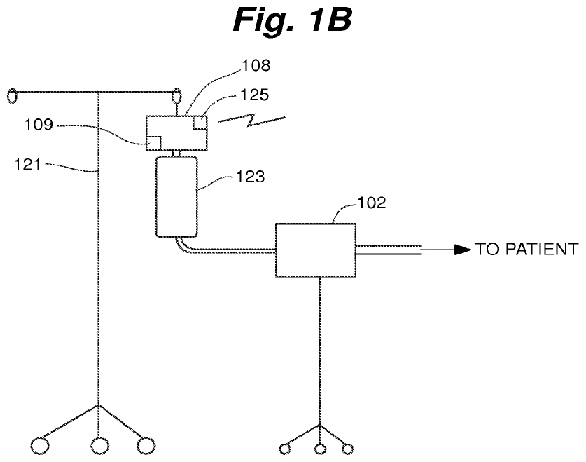

Referring to FIG. 1A, an exemplary nutritional monitor system of the present disclosure is shown generally as monitor system 100. Monitor system 100 includes one or more enteral pumps 102, a monitoring processor assembly 101, and one or more measurement capture devices 108. The measurement capture device 108 measures an entity related to the amount of formula output by enteral pump 102 and outputs measurement data, or alternatively, receives or captures measurement data related to an amount of formula output from the pump 102 from another device.

The measurement capture device 108 can be physically integrated into the IV pole on which the feeding bag and/or bottle is mounted and monitors the weight of the enteral nutritional formula bag or bottle.

The monitor system 100 can also include one or more local user devices 112 and/or one or more global user devices 120 that communicate with the processor assembly 101 for exchanging data and providing user access. Additionally, the processor assembly 101 can receive sensor data related to a patient and/or their pump 102 from one or more sensor devices 116, and/or identification data from one or more scanner devices 110.

Scanner device 110 can include an optical code scanner (e.g., a barcode scanner), a magnetic stripe reader, an RFID reader, etc., that can be used to read an identification code associated with, for example, a patient, a medical practitioner, the pump 102, packaging of a formula container (e.g., bag) of enteral or parenteral formula, and/or packaging of an oral nutrition item (e.g., food). The packaging of an oral nutrition item can include ID information as well as amount information that describes the amount of the oral nutrition item associated with the package. The amount can be described, for example, by portion size, weight, volume, and/or dimensions. The term “oral nutrition item,” as used herein, refers to an item of food or drink that a patient can consume. For example, a cup of juice, a bottle of baby formula, a slice of bread, an adult portion of chicken, an ounce of cheese, etc. Scanner device 110 can be a standalone device that is physically remote from other components of monitor system 100. In one embodiment, scanner device 110 can be physically and/or electronically integrated with user device 112 or enteral facility server 104.

In one embodiment, the measurement capture device 108 and the pump 102 can each be distinct devices, e.g., that mechanically and electronically operate independently, do not share physical or electrical components, and/or are housed in separate housings. Alternatively, the measurement capture device 108 and the pump 102 can be integrated physically and/or electronically. When physically integrated, the measurement capture device 108 and pump 102 can be housed within the same housing and/or share one or more physical components. When electronically integrated, the measurement capture device 108 and pump 102 can share one or more electrical and/or processing components or resources and/or be controlled by a shared controller device.

The monitor system 100 can operate with and be compatible with one or more vendor-neutral pumps 102, without restriction to a specific manufacturer or vendor, or proprietary design principals. In another embodiment, the pump 102, processor assembly 101, local user devices 112, and global user devices 120, can be configured to exchange data, such as in accordance with one or more predetermined data formats and/or protocols. The term “exchange,” as used herein, includes receiving and/or transmitting data.

The pump 102 includes a pump device that pumps formula for delivery of formula held in a formula container to a patient. The formula container holds a reservoir of formula to be pumped by the pump 102. The formula container can be configured, for example, as a bag or bottle. The pump device can be, for example, a volumetric pump with a rotary peristaltic pumping mechanism, or the like. The pump 102 can further include a control panel with a user interface, including a display and a user entry device. The display displays information about the output of pump 102, such as the volume of formula that has been pumped, and/or alerts. The user entry device includes, for example, a touch screen or keypad via which a user can enter information.

The pump 102 can further be provided with a controller 103 for controlling the pump device, such as for enabling and controlling the rate or amount of formula output by the pump 102. The controller 103 can be in data communication with an internal or external processor, such as via wired or wireless communication. Wireless communication can use, for example, radio waves, microwaves, infrared, ultrasound, or electromagnetic induction. Accordingly, control signals can be generated by processor assembly 101 to control operation of the pump 102. Additionally, the pump 102 can transmit information about operation of the pump 102 to the processor assembly 101, such as changes to formula administration pump settings (e.g., changed manually or by another processing device), or indicators (e.g., that provide notification of a blockage or interruption).

The pump 102 can be used to administer enteral formula to patients that are unable to normally ingest food due to, for example, the inability to swallow. The term administer, with reference to formula administration refers to outputting the formula so that it flows toward the patient, e.g., via a catheter. The pump 102 can administer enteral formula to a patient via an implantable or temporary enteral access device, such as a naso-gastric feeding tube (NGT), a percutaneous endoscopic gastrostomy (PEG) device, and the like.

In one embodiment, the measurement capture device 108 is mounted to or supported by an intravenous (IV) pole 121. The IV pole can support the enteral pump 102 and/or the formula container 123 that holds a reservoir of formula to be pumped by the pump 102. Alternatively, the measurement capture device 108 can be mounted to or supported by the pump 102, the formula container, and/or IV tubing that exits pump 102 for delivering formula to a patient's digestive system, such as via a nasal passage or a surgically established passage to the stomach or jejunum. Alternatively, the measurement capture device 108 can be mounted to or supported by a different platform than the IV pole.

In one embodiment, the measurement capture device 108 is configured to capture the weight of the formula container by measuring the weight. In this embodiment, the measurement capture device 108 can be supported by the IV pole 121, such as suspended from an arm or hook that extends from the IV pole. The formula container can be suspended from the measurement capture device 108. The measurement capture device 108 includes a device that measures the weight of the formula container suspended from it. For example, the measurement capture device 108 can include a scale, a strain gauge, and/or electrical components that output analog or digital weight data that indicates the weight of the formula container. In this embodiment, the measurement capture device 108 outputs the weight data as measurement data.

Other embodiments of measurement capture device 108 are envisioned, such as an embodiment in which the measurement capture device 108 supports the formula container from underneath the formula container, measures its weight, and outputs corresponding measurement data.

In addition to a digital or analog scale for measuring weight and outputting measurement data, the measurement capture device 108 can include a communication device 125 for transmitting the measurement data via wired or wireless communication, a battery, and/or an indicator device, such as an LED light or audio alarm that can indicate when battery power is low or when an interruption in delivery of formula to the patient has occurred (e.g., due to no change in measured weight for a threshold time interval).

Since weight measurement devices are reliable, accurate, and easily calibrated, they are well suited for monitoring formula delivery to a patient. Additionally, the measured difference in weight of the formula container is the weight of formula that actually exited the formula container and was delivered to the patient.

In another embodiment, the measurement capture device 108 includes a flow meter, or the equivalent, that measures a rate of flow of formula that exits the pump 102. In this embodiment, the measurement capture device 108 outputs the measured rate of flow as measurement data.

In still another embodiment, the measurement capture device 108 includes a camera that captures an image of the display of pump 102, and outputs image data. In this embodiment, the measurement capture device 108 outputs the image data as measurement data. In this embodiment, the measurement capture device 108 captures the weight information by acquiring and processing the image of the data displayed by the pump 102, and determining the weight from the displayed data.

The measurement capture device 108 can include a clock device that timestamps the measurement data to indicate when the characteristic measured or captured was actually measured or captured. Alternatively or additionally, the measurement data can be timestamped by a clock device included with the processor assembly 101.

The measurement capture device 108 can include a processor or controller 109 to control operation of the measurement capture device 108 and/or to process the measured or captured data so that the measurement data output by the measurement capture device 108 has been processed. Processing of the measured or captured data can be performed by processor 109, the processor assembly 101, another processor, or a combination thereof.

When the measurement capture device is configured to measure the weight of the formula container, processing the weight data can include determining the weight of formula that exited the formula container and was delivered to the patient over at least one selectable time interval by determining the change in the weight of the formula container. The processing can further include monitoring the change in weight over a series of time intervals. Additional processing can include determining the volume of formula that corresponds to the weight of formula that was delivered over one or more time intervals. The user can select settings associated with the processing, such as the duration of the time intervals, the number of time intervals in between processing the measured weight data, and the number of time intervals in between monitoring the weight or volume of exited formula.

When the measurement capture device is configured to measure the rate of flow of the formula that exits the pump 102, processing the rate of flow can include determining a rate of flow over at least one time interval. Additionally, the rate of flow information can be used to determine a volume of formula that flowed from the pump 102 and was delivered to the patient over one or more time intervals.

When the measurement capture device is configured to capture an image of the display of the pump 102, processing the captured image data can include interpreting the image data, which can include processing the image data using optical character recognition (OCR). The image data processed with OCR can be further processed to determine an amount, e.g., volume, of formula that exited the pump 102 over at least one time interval. The displayed information that was captured in the image can include, for example, a rate of flow, interruption in flow, on/off/hold status of the pump 102, and an occurrence of an alarm. If the rate of flow was displayed as “00.00,” the status indicated was “off” or “hold,” or an alarm was activated, a time period can be determined for which formula was not administered to the patient.

In another embodiment, the measurement data can be entered into the processor assembly 101 manually by a user, e.g., via a user input device, such as a keyboard or touch screen. In a further embodiment, the measurement data can be received by the processor assembly 101 directly from the pump 102, either in real-time or after having been stored for a period of time.

When the nutrition administered to the patient is oral nutrition, the nutrition (the type of food or supplement) and amount (e.g., volume, serving size, weight) that was served or fed to the patient and was ingested, or an estimate thereof, is input to the processor assembly 101, such as by user entry or by scanner device 110.

The processor assembly 101 can thus determine how much of a nutrient was administered to the patient using nutrition administration information and nutritional information for the formula or oral nutrition that was administered. The nutrition administration information can include the amount (which can be based on the received or entered measurement data) and the type of formula (which enteral or parenteral formula) or oral nutrition (which food) that was administered to the patient. The nutritional information for the formula or oral nutrition administered to the patient can be accessed by consulting nutrition database 118. The processor assembly 101 can further access one or more databases systems 114a-c and 118. Database systems 114a-c store information about the patient, and other patients, such as demographic information, nutritional history information, diagnostic information (e.g., laboratory result and imaging data, etc.), and allergy information. Database 118 stores information about nutrition that can be administered to a patient, such as enteral and parenteral formula and oral nutrition items. The term “administer” with reference to an oral nutrition item (food) refers herein to serving the food or feeding the food to the patient in order that the patient ingest the food. The term “access,” as used herein, can include receive and/or store.

The processor assembly 101 can continually, or upon request, monitor, store, and analyze the measurement data associated with a patient, forming a record of the patient's daily and long term nutritional intake. The nutritional record can be updated during or after nutrition is administered to the patient. For example, the nutritional record can be updated immediately after (e.g., in real-time), soon after (e.g., in near real-time, with a small lag), or well after (e.g., at the end of a nurse's shift) the nutrition is administered.

The processor assembly 101 processes the received measurement data to determine the patient's nutritional intake. Nutrition, as used herein, refers to any combination of nutrients. Nutrients, as used herein, refers to components of nourishment, such as calories, macronutrients, micronutrients, proteins, fats, carbohydrates, vitamins, minerals, and electrolytes amounts (ex. potassium, magnesium, calcium, and phosphorous), without limitation thereto. Nutritional information about the enteral formula, parenteral formula, and oral nutrition administered to patients can be accessed from nutrition database 118 and can be used, in addition to the measurement data, to determine the patient's nutritional intake.

Identification of the patient and the measurement capture device 108, the nutrition administered (e.g., a package or container associated with administered enteral formula, parenteral formula, and/or oral nutrition) can be provided to the processor assembly 101 by user input or by the scanner device 110. For example, an optical code (e.g., barcode or matrix optical codes) or RFID tag encoded with an identification code provided with the patient and/or bag of formula can be scanned by the scanner device 110. Scanner device 110 provides the scanned identification code to the processor assembly 101.

The processor assembly 101 can access information about the patient from digital information systems, such as databases 114a-c, other computer systems, and/or user input. Such information can include, for example, demographic information, the patient's electronic medical record (EMR) (e.g., including medical history and current health status), and diagnostic results from testing performed on the patient.

The nutrition database 118 can also be consulted to access the nutritional content of the enteral formula provided to the patient and determine the patient's nutritional intake. The processor assembly 101 can further compare the patient's nutritional intake to predetermined nutritional requirements. The term “nutritional requirements” as used herein refers to the nutritional needs of the patient for one or more nutrients, based on factors, such as the patient's weight, age, and health status. This comparison can be done in real-time, near real-time or at some other time after the formula was administered. An analysis can be performed that compares formulas and determines the optimal formula for the patient in view of the patient's medical and nutritional status and history.

The processor assembly 101 compares the patient's nutritional requirements to the patient's nutritional intake associated with a selectable time period. The patient's nutritional requirements are satisfied if there is not a substantial difference between the patient's nutritional requirements and the patient's nutritional intake over a predetermined time interval. A substantial difference occurs when the difference exceeds a predetermined threshold, which can be a relative or absolute value determined by a medical professional. The substantial difference can be an amount that could affect the patient's health status.

If there is a substantial difference, the processor assembly 101 determines an adjustment, which can include changing the formula to be administered or administration parameters for the formula. Administration parameters include, for example, administration rate (weight/time or volume/time), duration, and/or total weight or volume, for a selectable number of time intervals. The parameters associated with each time interval can be the same or different. The adjustment can be implemented by providing a supplemental bolus or a series of supplemental boluses of the selected formula using selected administration parameters. Alternatively, the administration parameters already being used for administering the formula can be adjusted, e.g., for each time interval or indefinitely. The adjustment can be continuous or intermittent, and it can be static or vary dynamically over time.

For example, when the measurement capture device 108 is configured to measure weight, the administration parameters determine the pump's 102 administration rate (weight/time), duration of administration, and/or total weight of formula to be administered, for a selectable number of time intervals. An adjustment can be calculated to adjust the administration parameters to compensate for a difference between the patient's nutritional requirements and the patient's nutritional intake.

The processor assembly 101 can generate control signals that are provided to the controller 103 of the pump 102 for controlling operation of the pump 102. Alternatively or additionally, processor assembly 101 can provide control signals to processor 109 of the measurement capture device 108 for controlling operation of the measurement capture device 108. For example, the processor assembly 101 can control the pump 102 to output the formula in accordance with selected administration parameters.

The processor assembly 101 can calibrate the pump 102 or the measurement capture device 108, such as by determining a difference between expected output data and actual output data. In one example, the actual output is the calculated weight, which can be based on a difference in the measured weight of the formula container over a time interval. In another example, the actual output is based on the volume of formula that exited the formula container, which can be determined from the measurement rate of flow over a time interval. The expected output data can be based on control input to the pump 102, such as user input settings or control signals to the controller 103 of pump 102, which specify a selected amount of formula that the pump 102 should output.

The processor assembly 101 calibrates the pump 102 by generating a control signal that controls the pump 102 to adjust the amount of formula to be output by the pump 102. The adjustment can be in accordance with the determined difference between the expected and the actual output data.

The processor assembly 101 calibrates the measurement capture device 108 by generating a control signal that controls the controller 109 of measurement capture device 108. The control signal can adjust the measurement data output by the measurement capture device 108 in accordance with the determined difference between the expected and the actual output data. Here, the expected output data can be a known weight for a formula container measured by the measurement capture device 108.

The processor assembly 101 can process the measured weight data to determine if the measured weight data indicates the occurrence of a movement event related to movement of the formula container or patient. For example, when the patient is transported or ambulates, and the pump 102 is transported with the patient, the movement of the pump 102 can affect the measured weight data. Similarly, if the formula container is manipulated, such as by a nurse, bedside clinician, or caregiver when changing the bag of formula or shaking it to eliminate an air pocket, the measured weight data can be affected as well. The change in measured weight data caused by such movement events should not be used in a determination of the formula administered to the patient or the nutritional intake of the patient. Thus, the processor can be configured to ignore changes in measured weight data related to such movement events when calculating patient intake. In an embodiment, a determination is made whether a detected movement of the formula container exceeds a predetermined event threshold in terms of degree of movement and/or duration. A determination that the event threshold was exceeded indicates that an event occurred. Once the movement stops, the scale is recalibrated and/or determines the current weight of the formula container. If the difference between the current weight or volume of the formula in the container relative to the last weight or volume measured before the event occurred is below a bag-change threshold, then the event is determined to be an artifact or a movement event that corresponds to movement of the formula container or the patient. If the difference in weight or volume exceeds the bag-change threshold, then a determination is made that a bag change event occurred. The movement event or bag change event can be stored and time-stamped.

The processor assembly 101 can use the information about the patient, as well as aggregated data about other patients with similar conditions or demographics (e.g., by consulting aggregated knowledge base 122), to perform data analysis, conduct research studies, make diagnoses, and make recommendations with respect to which formula to administer and selection of optimal administration parameters. Additionally, the processor assembly 101 can make predictions, including calculating a change in patient nutritional requirements in response to an update to patient information, or determining the medical effect of an update to formula or administration parameters. The term “updates,” as used herein, can include actual changes, theoretical changes, or receipt of an initial value or selection.

The local user device(s) 112 and global user device(s) 120 include computing devices, such as a portable device (e.g., smartphone, personal computing device (PDA), laptop, or tablet); a personal computer (e.g., desktop); and/or a workstation of a computer network. The local user devices 112 and global user devices 120 can receive information via a user interface or an integrated device, such as a camera or optical code scanner. Additionally, the local user devices 112 and global user devices 120 can exchange information with the processor assembly 101. In an embodiment, the local user devices 112 and/or global user devices 120 can perform some or all of the processing functions that are performed by the processor assembly 101 in addition to, or instead of, the processor assembly 101 performing such functions.

In one embodiment, a local user device 112 receives data, via wired or wireless communication, associated with a patient or with several patients, including ID data that is output by one or more scanner devices 110, measurement data that is output by one or more measurement capture devices 108, and/or sensor data that is output by one or more sensor devices 116. Alternatively, the ID data, measurement data, and/or sensor data can be entered manually via a user input device associated with the local user device 112. For example, a local user device 112 can be provided at each patient's bedside, in a patient's room that can accommodate multiple patients, or at a nursing station that services multiple patients and/or patient rooms. The ID data is used to correlate the received measurement data and/or sensor data to a particular patient, pumps 102 or 124, formula container, and/or measurement capture device 108. The local user device 112 can additionally, or alternatively, be used to enter additional data, such as user entered data or digital data from one or more devices, e.g., a medical practitioner's observations or assessments, the patient's weight, the patient's vital signs, etc.

The local user device 112 can process the ID data, measurement data, and/or sensor data and/or transmit the data to the enteral facility server 104. Processing of the ID data, measurement data, and/or sensor data can be performed by the local user device 112, the enteral facility server 104, or a combination thereof. In an embodiment, the local user device 112 can access the databases 114a-c. Additionally, the local user device 112 can exchange data with other local user devices 112.

The monitoring processor assembly 101 is shown to include at least one computing device, which in the current embodiment includes at least one enteral facility server 104 and enteral web server 106. The functions and components described and shown for the servers 104 and 106 are provided to describe an example embodiment, but are not limited hereto, however in other embodiments, these components and functions can be distributed or combined.

The enteral facility server 104 can include one or more computing devices that can be physically and/or electronically integrated together, and/or can be positioned physically remote from one another. The functions of enteral facility server 104 can be distributed among the computing devices. Additionally, two or more computing devices can perform the same functions.

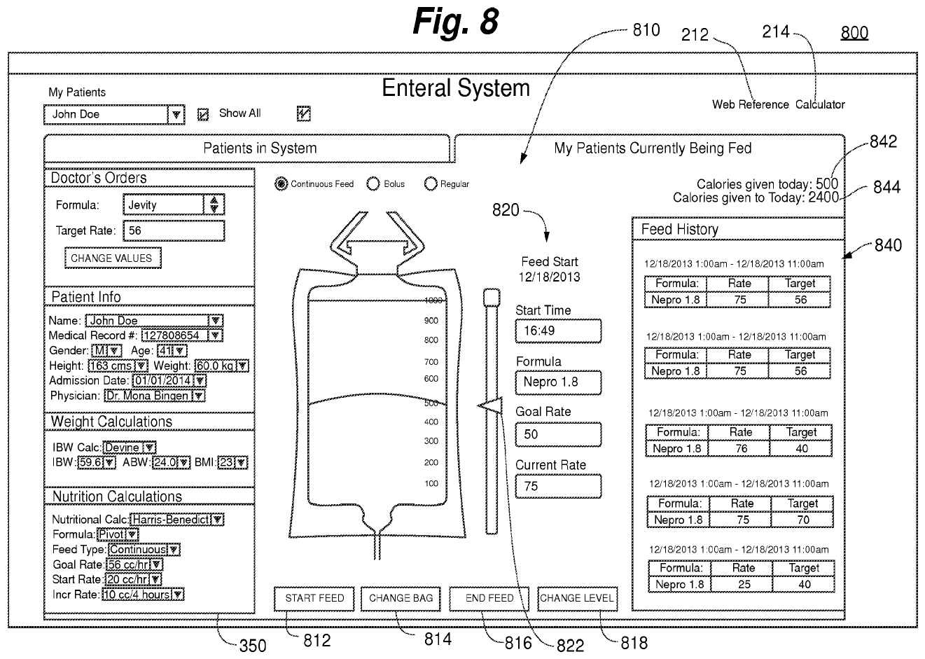

The enteral facility server 104 receives identification (ID) information from scanner devices 110, authorized user devices 112, 120, or via a user input device of the enteral facility server 104 (shown in FIG. 8). The identification information can include a patient's ID that identifies the patient, a pump ID that identifies the pump 102, a measurement capture device ID that identifies the measurement capture device 108, and/or a formula container that identifies the bag or bottle containing the formula that the pump 102 is administering.

The enteral facility server 104 also receives information from database systems, including a Healthcare Information System (HIS) 114a that stores patient demographic and medical record information for a plurality of patients, a Laboratory Information System (LIS) 114b that stores results from diagnostic tests performed on a plurality of patients (e.g., laboratory, radiology, imaging, EEG, EKG tests), and an Allergy Information System (AIS) 114c that stores allergy history information about a plurality of patients. The received database information can be used by the monitoring processor assembly 101 during its analysis processing.

The enteral facility server 104 can receive sensor data from sensor device(s) 116 that sense characteristics of the patient. The sensor devices 116 can include a sensor placed internally or externally to a patient, such as for sensing a volume of contents within the patient's stomach. Sensor devices 116 can further include one or more sensors for sensing changes in metabolic rate or CO₂ consumption, using, e.g., indirect calorimetry, spirometry, respiration exchange ratio (RER), and/or capnography. The received sensor information can be used by the monitoring processor assembly 101 during its analysis processing.

Enteral web server 106 communicates with enteral facility server 104, e.g., via wired or wireless communication, or a combination thereof. The communication can be via a network, such as the Internet, an intranet, or a LAN. The enteral web server 106 can be a server that operates in the cloud such that it is remote from server 104 and exchanges data via the Internet. The enteral web server 106 can be an Internet based hub that communicates with multiple enteral facility servers 104 located at different facilities (e.g., hospitals and nursing homes, long term care facilities, and home use), provides a web interface for the facility servers 104 and for users. The enteral web server 106 can host a web site that is accessible by users via user devices 112 and/or 120 and enteral facility servers 104. Via the website, facilities and users can access data stored about patients or formulas, request analysis to be performed, update software applications, etc.

Enteral web server 106 can exchange information with one or more user devices 112 or 120. The user device(s) 120 are computing devices that can be configured, for example, as one or more devices, such as a portable device, e.g., a smartphone, personal computing device (PDA), or tablet; a personal computer; and/or a computer terminal of a computer network. The user devices 120 can be used to capture information, receive information, and/or input or receive information to and from the enteral web server 106.

User device 112 and user device 120 can be integrated as one device. Since user device 112 is used to access the enteral facility server 104 and user device 120 is used to access the enteral web server 106, an integrated user device 112/120 may need to use different software portals and provide different authorization indicia for accessing each of servers 104 and 106. In an embodiment, servers 104 and 106 can use a single access portal that they share, such that a user who has gained access via the access portal can access either server 104 or 106, although additional authorization information can be needed for accessing servers 104 and 106. Similarly, in one embodiment, the same software application (or app) can be used by a single device 112/120 to access both servers 104 and 106. In another embodiment, different software applications (or apps) may be needed to access each of the servers 104 and 106. It is envisioned that both apps can be stored and operated on a single device 112/120. Each user device 112, 120 and/or each user of the user devices 112, 120 can have different authorizations for entering, updating, or accessing information.

Servers 104 and 106 can receive information from nutrition database 118 about a variety of formulas, including nutritional content, ingredients, availability, cost per unit, storage requirements, entitlement for insurance coverage by various insurance companies, etc. The nutrition database information can be used by the monitoring processor assembly 101 during its analysis processing. Additionally, the web servers 104 and 106 can access information in aggregated knowledge base 122, including aggregated information about a plurality of patients related to nutrition administration to the patients. The aggregated information can be clustered so that servers 104 and 106 can locate data among the aggregated data for patients that are similar to a particular patient.

The functions and components described and shown for the enteral facility server 104 and the enteral web server 106 are provided to describe an example embodiment, but are not limited hereto. In other embodiments, these components and functions can be distributed or combined. Additionally, information can be exchanged between servers 104 and 106. Since servers 104 and 106 can access much or all of the same data, they have the capability of performing at least some of the same functions.

Communication between components of monitor system 100, including 101, 102, 103, 104, 106, 108, 109, 110, 112, 114a-c, 116, 118, 120, 122, and 124 can be via wired or wireless communication, or a combination thereof. Some or all of such communication can be via one or more networks, such as the Internet, a LAN, WAN, cellular network, etc.

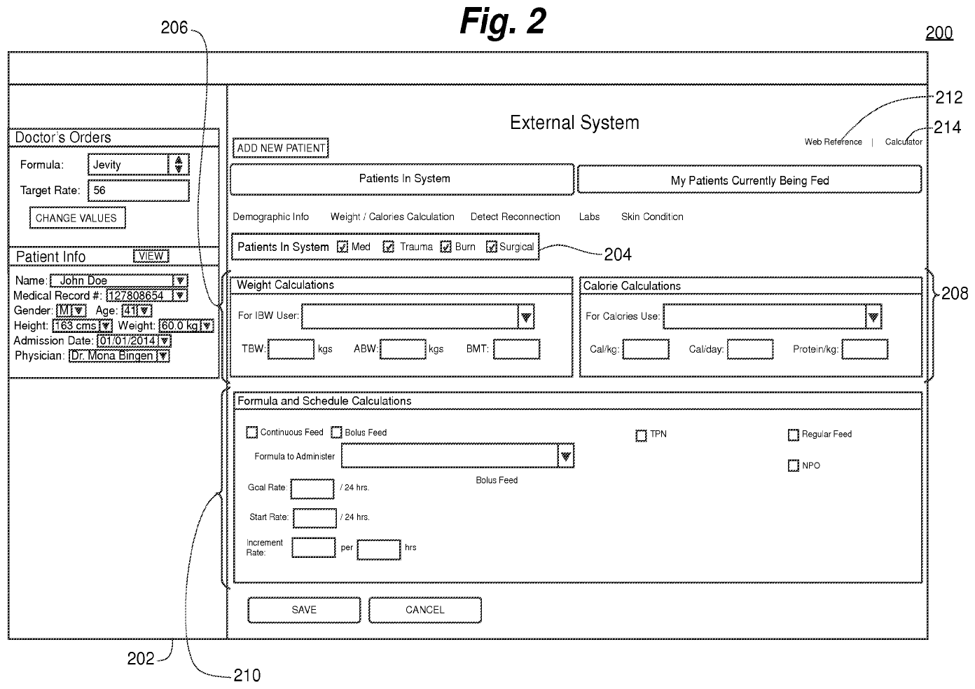

With reference to FIGS. 2-10, example screen shots provided by a graphical user interface (GUI) are shown. The processor assembly 101, enteral facility server 104, enteral cloud server 106, local user devices 112 and/or global user devices 120 can provide the GUI, store information that is entered via the GUI, and store and calculate information that is displayed via the GUI. The GUI is displayed on a display device and can receive input data from a user input device, such as a keyboard, point-and-click device (e.g., mouse), microphone, touch screen, etc. A user can change between different screens and views, such as by operating a tab provided on the GUI. The user can select to view the screens shown in FIGS. 2-10 as well as other screens, such a screen showing the patient's vitals, the patient's demographic information, the patient's EKG, medical historical information for the patient, etc.

FIG. 2 shows the patient's nutritional requirement information displayed in screen 200. Screen 200 includes a patient demographic area 202, patient status area 204, weight calculation area 206, nutritional requirement calculation area 208, and formula and formula parameters calculation area 210. The formula parameters can include a formula selected for administration and/or administration parameters.

Patient demographic area 202 includes demographic information related to a selected patient who is identified in the current example as Rumson Crown as well as by an electronic medical record (EMR) identification number. The demographic information can be retrieved from the patient's EMR stored in the HIS database 114a. The demographic information can include, for example, birthdate, age, gender, height, admission date, and physician name. Additional patient information can be included, such as allergy information, which can be retrieved from the AIS database 114c. The demographic information can further include patient status information, such as the patient's health status (e.g., a specified disease, gastric condition, heart condition, smoker, etc.); prescribed medication; prescribed medical intervention (e.g., surgery, physical therapy, etc.); diagnostic result (e.g., laboratory, radiographic, or related diagnosis results); activity level; feedback (about, e.g., energy level, gastric comfort, etc.); caregiving level available; place of residence (e.g., a move back home, to a hospital, nursing home, intensive care unit, etc.); patient financial means; patient insurance coverage; and patient preference of formula.

The demographic information can be entered by the user, imported from the HIS database 114a, the AIS database 114b, or the patient's EMR, etc. Information that is imported can be imported upon a user request, at scheduled intervals, or upon occurrence of an event, such as when data is updated.

Weight calculation area 206 includes weight related information about the patient, including, for example, admission weight, current weight, ideal body weight (IBW) or adjusted body weight (ABW). The weight information can be user entered or imported from another device or database. Equations to estimate IBW and ABW such as the Devine formula, Robinson formula, Miller formula, Hamwi Formula, Adjusted body weight equations can be selected, e.g., by the provider or automatically.

Nutritional requirement calculation area 208 includes a nutrition consumption calculator equation, which is used to determine estimated nutritional requirements (e.g., goals). The goals include, for example, a daily caloric goal, calories per kilogram, calories per kilogram per day, and protein per kilogram. Other goals can be determined as well, such as for other nutrients and time intervals other than daily time intervals. The nutrition consumption calculator equation can be selected from equations such as the Harris Benedict equation, Mifflin-St Jeor equation, Ireton-Jones Energy equation, Currei formula, Weir equation, Owens equation, and the like. The provider can select an appropriate equation based on the patient's medical status, e.g. medical trauma, surgical, or burn, mechanical venting support. In an embodiment, the nutrition consumption calculator equation is selected automatically, or a limited set of selections that the practitioner can choose from are determined automatically, e.g., by one of servers 104 or 106, based on the patient's medical status. In an embodiment, automatic selection can be overridden by user selection or facility or institution preferences. The nutritional goals can be calculated based on the patient's height, weight (e.g., IBW or ABW), and stress factors. The patient's weight measurement that is used for calculating the nutritional goal per time period can be highlighted.

Formula and Schedule Calculations area 210 displays information such as the enteral formula selected, formula administrative parameters, the feed type (continuous, bolus, bolus duration, enteral or parenteral, or orally administered nutrition), the enteral goal rate, a start rate for administration to start at, and an increment rate. The rates can be described in terms of volume or nutrient amount per a time interval, such as calories per day. The start rate can be lower than the goal rate to gradually accustom the patient to administration of nutrition in the current form. The start rate can also be higher than the goal rate to jump start the administration, or transition from a previous administration session having a higher rate than the current session. The increment rate is an amount by which the administration rate can be adjusted per a selected time increment to adjust the administration rate from the start rate to the goal rate during the administration session. Some of the data fields, e.g., formula selected, feed type, and start rate, can accept user entry data by an authorized user. When one data field is updated, the other fields can then be calculated and updated in accordance with the patient's nutritional requirements.

The demographics and physical characteristics of patients having data stored in the databases 114a-114c can be analyzed and correlated with nutrition information. Trends can be determined. For example, it can be determined when patients having similar demographic, physical, or diagnostic characteristics respond well or poorly to a nutrition plan that has specific nutritional requirements.

Determination of the patient's nutritional requirements can include accessing information stored in the HIS database 114a and the LIS database 114b, including information about stress factors. For example, the patient's nutritional needs can be adjusted when a lab result or a medical condition indicates that the patient has a deficiency or surplus that can be corrected by adjusting the patient's nutritional requirements. For example, when the patient's medical records indicate that the patient has osteoporosis or a family history of osteoporosis, the patient's nutritional requirement for calcium can be increased. When the patient has a history of iron deficiency, the patient's nutritional requirement for iron can be increased. However, when the patient has a medical condition of constipation, the patient's nutritional requirement for iron can be decreased.

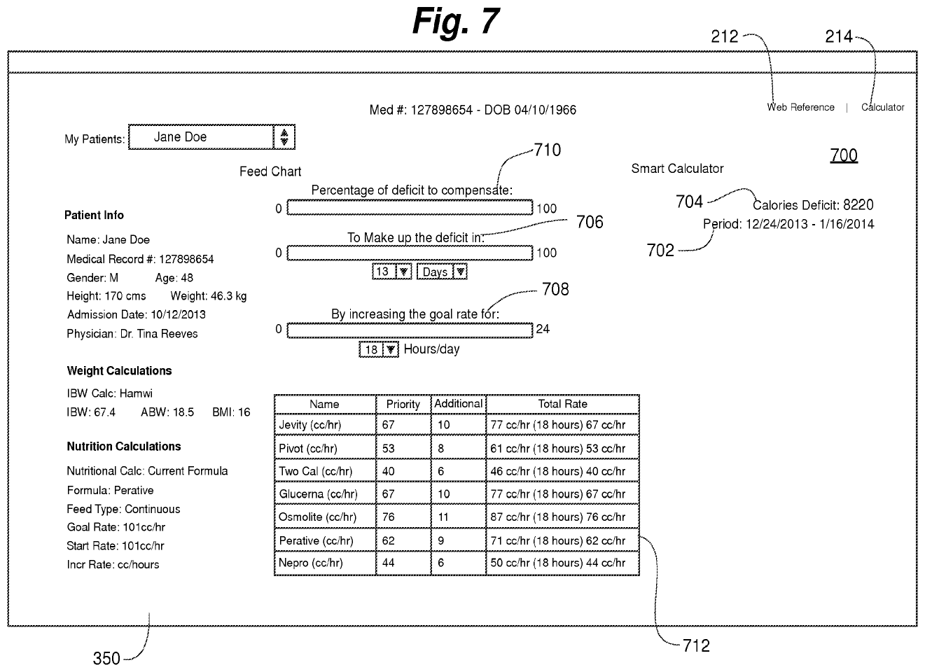

Tab 214 provides access to a compensatory calculator that is used to predict or determine nutritional compensation for an accrued nutritional deficit (see screen 700 in FIG. 7). This can include determining a nutritional surplus or deficits accrued over a specified time period relative to the nutritional goals, and suggesting compensation over specified time.

Tab 212 provides access to a web reference library that includes several links to websites that provide information on patient nutrition. The information can be updated and augmented. Examples of information include recommended nutritional guidelines, such as provided by websites of the American Society of Parenteral and Enteral Nutrition, the European Society for Clinical Nutrition and Metabolism, and the US Department of Health National Heart Lung and Blood Institute. Additionally, the websites can include clinical sites in which nutritionally related data can be collected, accessed, and analyzed. The websites linked to can further include websites of suppliers of enteral nutrition formulas and supplements.

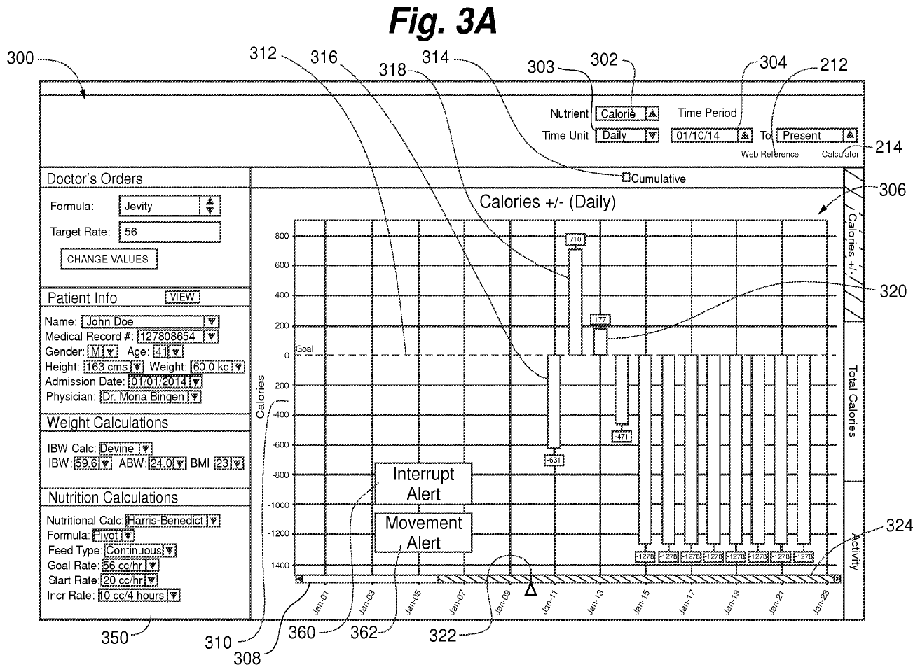

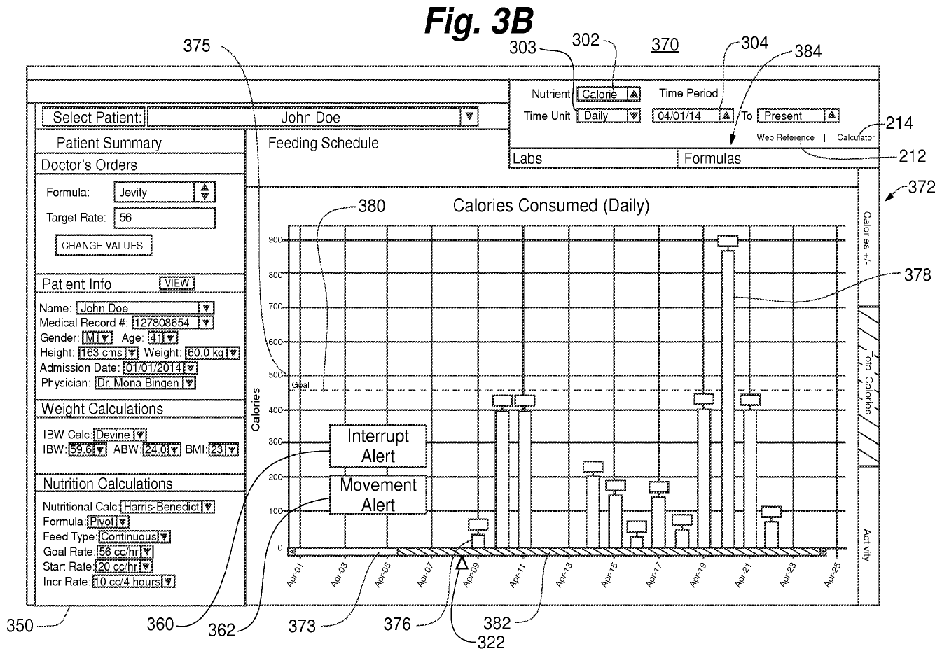

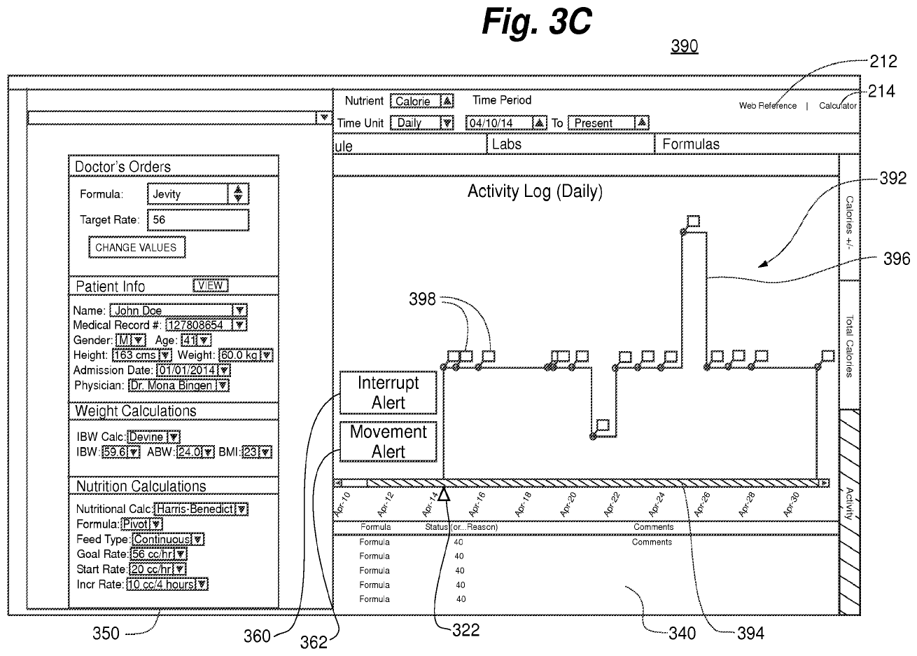

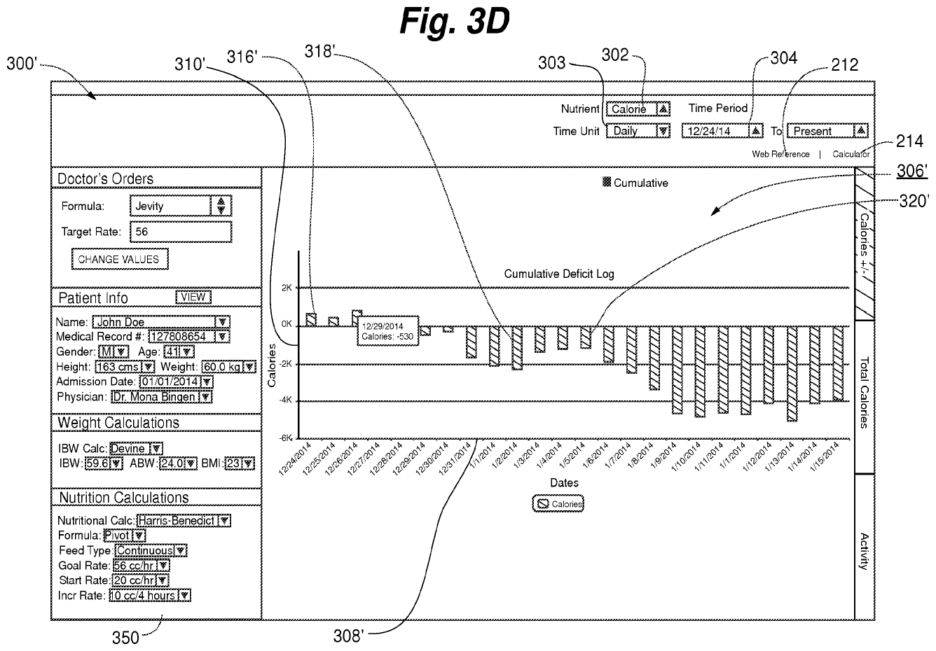

With reference to FIGS. 3A-3C, the GUI displays historical and/or predictive nutritional information for a selected patient. Screens 300, 370, 390, and 300′ shown in FIGS. 3A, 3B, 3C, and 3D, respectively, display nutritional history information for the selected patient based on information about the patient's nutritional intake that is recorded and stored over time. The nutritional history information can be based on measurement data received from the measurement capture device 108, measurement data related to the administration of enteral formula, data from scanner device 110, nutritional information retrieved from nutrition database 118, and/or user entry screen 300 shown in FIG. 3A shows a log of incremental (e.g., daily) deficit/surplus for a selected nutrient administered to the patient over a selected time period. Screen 300′ in FIG. 3D shows a log of incremental (e.g., daily) cumulative deficit/surplus for a selected nutrient administered to the patient over a selected time period. Screen 370 in FIG. 3B shows a log of incremental (e.g., daily) amounts of a nutrient entered over a selected time period. Screen 390 in FIG. 3C shows a log of incremental (e.g., daily) activity for a selected nutrient administered to the patient over a selected time period.

The log shown in screens 300, 370, 390, and 300′ can show data for past, present, or future time, or a combination thereof. The selectable time period can include time that has passed, so that the log shows a history of information for the selected nutrient. The selected time period can further include a future time, so that the log shows a prediction of information for the selected nutrient. Additionally, the selected time period can include a present time, so that the log shows information for the selected nutrient as it is updated in real-time or near real-time.

The nutrient can be selected using tab 302. Tab 302 can be operated to display a drop-down menu from which the provider can select the nutrient and unit (including macronutrient or micronutrient) for which data is displayed. In the example shown, the selected nutrient is calories.

Tab 303 can be operated to select a periodic time unit. The periodic time unit selected in screens 300, 370, and 390 is daily. Other periodic time units that be selected include, for example, hourly, per shift (e.g., an 8-10 hour shift), and weekly.

The time period can be selected, for example, using time period selection tabs 304 or time period selection controls 322 that slide along horizontal slide bar 324. The horizontal slide bar 324 can scroll horizontally along the time axis, and time period selection controls 322 can be slid to include past time, the present time, and/or a future time. In the example shown in FIG. 3A, the time period selection controls 322 only indicate the beginning of the selected time period. Accordingly, the selected time period extends to the present time and is updated in near real-time. For example, an update can be performed at predetermined time intervals, such as once per time unit at a predetermined time.

Graph 306 is plotted along a horizontal time axis 308 and a vertical nutrient value axis 310. The selected time units are indicated along axis 308. The example graph 306 shown is a step graph having a bar for respective time unit intervals (e.g., daily) wherein the vertical height of each bar represents the level of a nutritional (caloric in the present example) deficit or surplus that has accrued over the corresponding time unit interval (e.g., day). The vertical height is measured from goal line 312 that represents the goal for the amount of nutrients to be administered daily. Accordingly, the deficits or surpluses are measured relative to the goal.

The deficit and surplus is determined by performing a nutritional analysis over a selectable time period for each time unit interval (e.g., day), comparing the patient's nutritional intake based on the measurement data to the patient's nutritional requirement for the corresponding time unit interval.

A sequence of bars are displayed, each corresponding to a particular day for a selected time range. A first bar 316 shows a daily caloric deficit of −631 for day 1. Bar 316 indicates that the patient did not meet his/her daily caloric intake for that day. Possible reasons for failing to meet the daily requirement can include late initiation of feeding, frequent interruptions, and/or patient intolerance to the enteral nutrition. A second bar 318 indicates that on day 2, the patient met his/her daily caloric requirement along with a surplus of +710 calories. A bar 320 indicates that on the day 3, the daily caloric goal was again met, with a surplus of +177 calories. The demonstrated surplus of calories administered as in this example was delivered to compensate for the caloric deficit accrued day 1, and to compensate for an anticipated future deficit.

FIG. 3D displays a bar graph that shows accumulated deficits and surpluses for a series of time unit increments shown over a selected time range. The user can operate cumulative tab 314 to view accumulated deficits and surpluses, as shown in FIG. 3A. In the example shown, bar 316′ indicates that a surplus has accumulated by the corresponding day. Bar 318′ indicates that ten days later a deficit has accumulated. Bar 320′ indicates that after an additional three days, the deficit has decreased by about 50%.

When the cumulative tab 314 is deselected, the bars of the step graph show the incremental deficit or surplus, which is the deficit or surplus for the corresponding time period, non-cumulatively.

The incremental and cumulative deficit and surplus information can be used to compensate the nutritional intake so that the incremental or cumulative nutritional intake is within an acceptable zone above or below the goal line 312 by a target time (e.g., date and/or time). Compensation determination includes determining whether or not to increase the nutritional intake to compensate for a deficit or surplus. Compensation determination parameters can be used to determine whether or not, and how, to compensate. Compensation determination parameters can include, for example, compensation (incremental or cumulative) thresholds. For example, the incremental deficit associated with a particular time interval can be compared to the incremental deficit threshold, and/or the cumulative deficit associated with a selected time range can be compared to the cumulative deficit threshold. If one or more of the incremental and cumulative thresholds is exceeded, the nutritional intake can be adjusted so that the deficit will decrease by the target date. The incremental and cumulative thresholds can be selectable.

Other compensation determination parameters, described in greater detail below, can include, for example, an accumulation time period for which a deficit or surplus can be determined, a compensation time period having a plurality of intervals (e.g., days) that each include a plurality of sub-intervals (e.g., hours), and a selectable portion that indicates a portion of the sub-intervals for each interval that the nutrition administration is adjusted by providing the compensation.

Similarly, compensating for a surplus includes determining whether or not to decrease the nutritional intake to compensate for a surplus. This can include comparing the incremental surplus associated with a particular time interval to an incremental surplus threshold, and/or comparing the cumulative surplus associated with a selected time range to a cumulative surplus threshold. If one or more of the incremental and cumulative thresholds is exceeded, the nutritional intake can be adjusted so that the surplus will decrease by the target date.

The determination whether to compensate for a deficit or surplus by adjusting the nutritional intake can be made in real-time, near real-time, or upon the occurrence of an event. This can include generating an indicator to alert a medical practitioner that compensation for a deficit or surplus may be needed. Examples of an event that may trigger a determination to compensate include receipt of a request to optimize nutrition therapy of the patient and/or to compensate for a deficit or surplus.

Additionally, a notification can be generated when a change in nutrition status related data associated with the patient that indicates a change in, or a risk factor associated with, a nutritional requirement of the patient. The nutrition status related data can be associated, for example, with the patient's medical condition, e.g. an electrolyte imbalance, acute kidney failure requiring hemodialysis, respiratory failure requiring ventilator support, acute blood loss anemia, infection, and/or septicemia (e.g., as indicated by laboratory or diagnostic results), and/or development or changes in the condition of a skin wound (e.g., pressure ulcer).

Examples of events that for which compensation may be recommended include an anticipated interruption in nutrition administration, such as due to a scheduled operative procedure, a scheduled medical test for which the patient is not allowed any orally administered nutrition (NPO), or other interruption by a bedside clinician/primary care giver e.g., to provide routine care to the patient. The predicted deficit or surplus can be used to adjust the nutritional intake before and/or after the anticipated event to compensate for the predicted deficit or surplus before or after the event, so that the daily nutritional intake is near the goal line 312, e.g., within an acceptable zone above or below the goal line 312, by a target date.

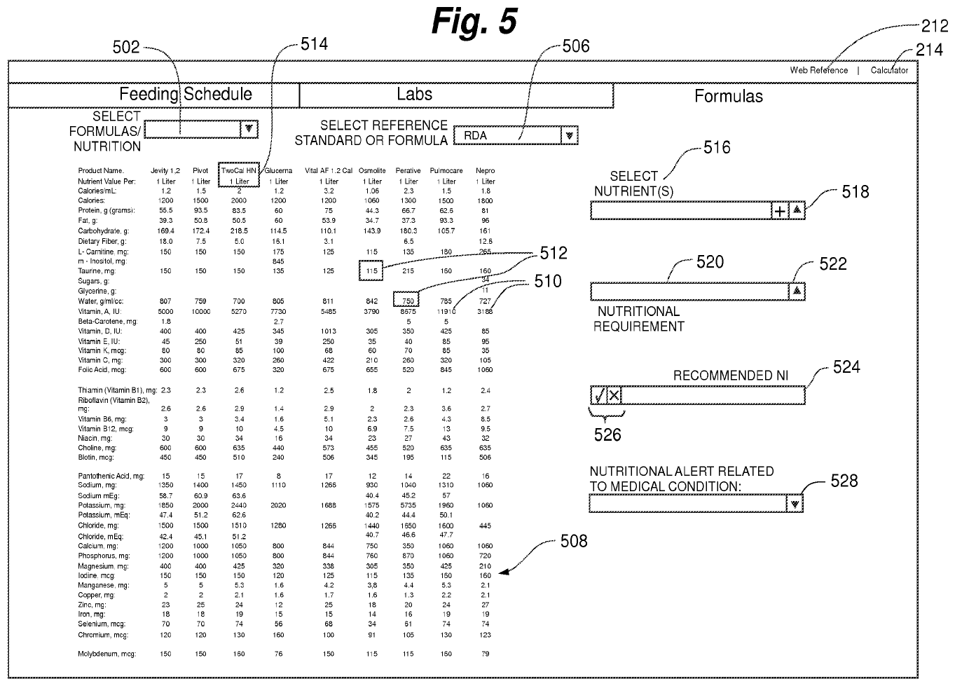

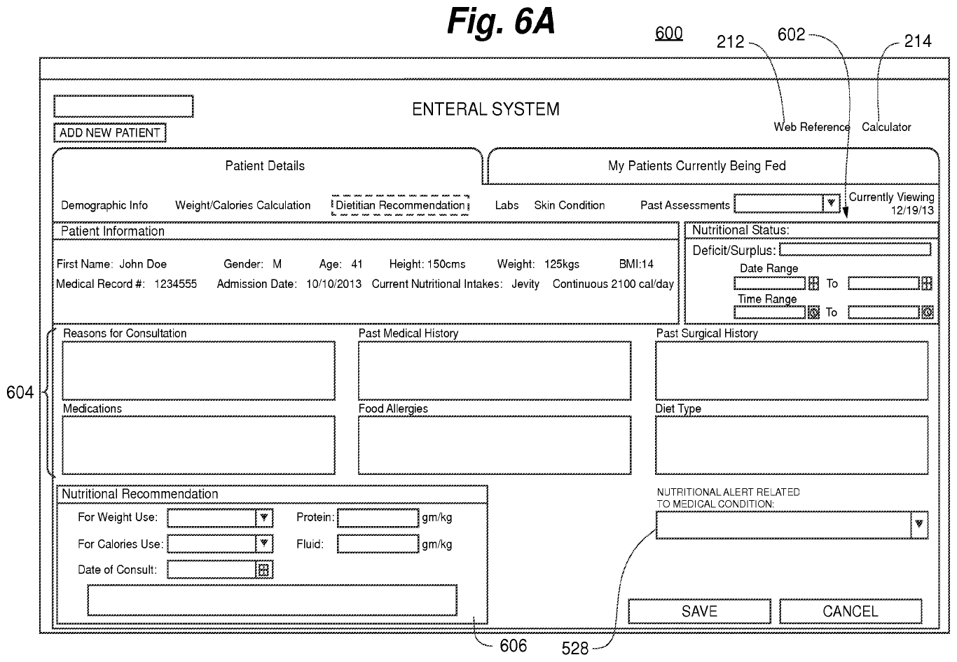

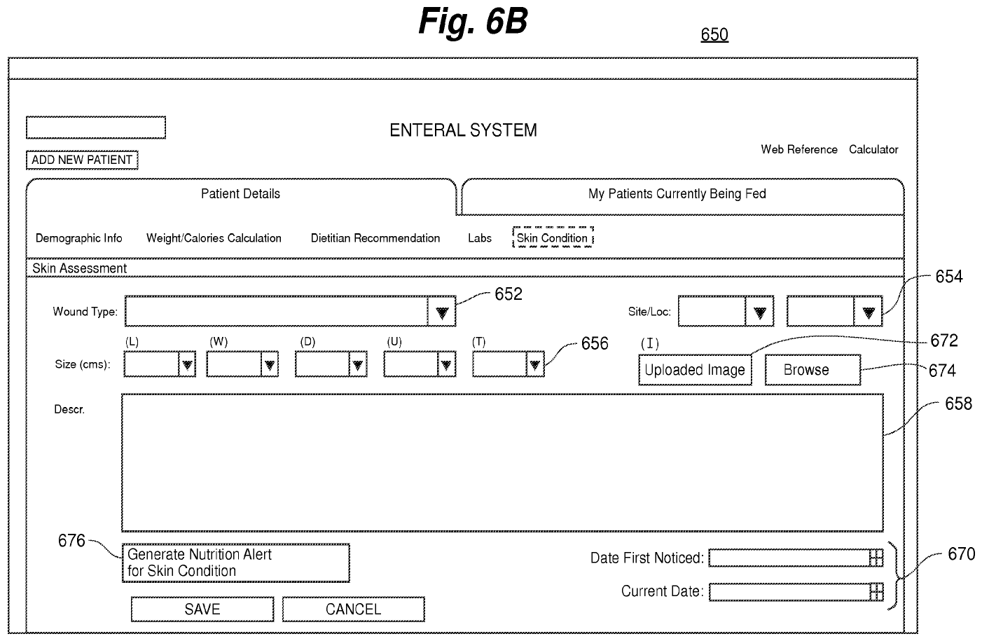

The information indicating changes in the patient's medical condition may be determined from information stored about the patient in the HIS 114a, such as in the patient's medical record or in another data storage accessible by the monitoring processor assembly 101. The information about changes in the patient's medical condition can thus be integrated with the nutrition administration information for the patient and the nutritional requirement data for the patient for determining the nutritional adjustment. Determining the nutritional adjustment can include determining when and/or how to adjust the administration of nutrition to the patient and/or notifying a medical practitioner and/or caregiver that an adjustment needs to be calculated or administered. The notification can include, for example, generating an indicator to be displayed on a screen of a GUI displayed by user devices 112 or 120 (see 528 in FIGS. 5 and 6A), generating a sound indicator via a user device 112 or 120, causing generation and transmission of an email, SMS text message, and/or voice message, etc.

Once a nutritional adjustment is determined, an indicator can be generated to alert a caregiver (e.g., a nurse) that an adjustment is being made or is recommended to be made. The indicator can be generated, and/or the nutritional adjustment can be made immediately when a determination is made, which can be in real-time or near real-time, or the nutritional adjustment can be scheduled.

The nutritional adjustment can include, for example, adding one or more boluses of enteral formula, changing the enteral formula based on a new medical diagnosis, adding supplemental nutrition, increasing vitamin intake, adding an oral nutrition item, adjusting the volume of a scheduled intermediate or bolus of enteral formula, and/or adjusting the administration rate of a continuous enteral or parenteral feeding for a selected time period. Adjustment to an administration rate can include a single adjustment or a series of adjustments, such as a gradual adjustment in which the change in rate is gradually (e.g., incrementally) adjusted over the time period. The adjustment can be stepped, such that it changes by stepped amounts over the time period.

With continued reference to FIG. 3A, when the user selects the time/date range to include a future time, an indicator can be provided to the user that alerts the user that predicted data for a future time is being displayed. The indicator can require the user to accept or decline viewing predicted data that corresponds to a future time. Additionally, a graphical or textual indication is provided that distinguishes the predicted data from present or historical data.

The predicted data is calculated using the assumption that the present formula parameters will continue to be used during the time period of the anticipated future event. Alternatively, theoretical formula or formula administration data can be entered and processed. Additionally, the predicted data is calculated using the patient's present nutritional requirements or predicted nutritional requirements for the patient. The patient's predicted nutritional requirements can be determined based on a prediction of the patient's response to the formula to be administered using the predicted formula administration parameters, a medical procedure planned for a future time, and aggregated data from aggregated knowledge base 122, including data about patients with similar demographic, medical, nutritional history, and/or nutritional requirements as the patient. Information about a medical procedure planned for a future time can be entered by a user, e.g., in response to a prompt for information, or can be retrieved from the HIS 114a.

Patient summary area 350 includes a summary of demographic, weight calculation, and nutrition calculation information about the patient. Patient summary area 350 can include an update feature that indicates when patient status information is updated that might affect nutrition calculations. If the provider is authorized, the provider can operate the view or edit buttons 352 to view or update the demographic, weight calculation, or nutrition calculation information.

FIG. 3B shows an example screen 370 of the GUI having a graph 372. Graph 372 is plotted along a horizontal time axis 373 and a vertical nutrient value axis 375. The nutrient and nutrient units indicated along the vertical axis 375 can be selected. Time units along horizontal time axis 373 can be selected to be, for example, minutes, hours, days, weeks, or months. The vertical height of each bar of the graph 372 shows the total amount of the selected nutrient that the patient has received for the corresponding time interval. The vertical height can be compared to goal line 380, which represents the goal for the amount of nutrients to be administered. Bar 376 is below goal line 380, indicating that there was a deficit at the corresponding time interval. Bar 378 is above the goal line 380, indicating that there was a surplus at the corresponding time interval.

The date and/or time range can be selected using time range controls (not shown) or horizontal slide control bar 382. The horizontal slide bar 382 can scroll horizontally along the time axis to include past time, the present time, and/or a future time.

FIG. 3C shows an example screen 390 of the GUI having a graph 392. Graph 392 is plotted along a horizontal time axis 394. Bars of the graph 392 show when the Activity display mode is selected, the administrated quantity of a selected nutrient/unit is shown over time, where time can be indicated in a selected unit, such as minutes, hours, days, weeks, or months. Graph 392 shows each event that occurred within the selected time interval. The events can include, for example, a change of an enteral feeding bag, an interruption, and a shaking of the IV pole. The date and/or time range can be selected using time range controls (not shown) or horizontal slide control bar 394. The horizontal slide bar 394 can scroll horizontally along the time axis 394 to include past time, the present time, and/or a future time. A time range control (not shown) can also be provided for selecting a time range.

Accordingly, FIGS. 3A-3C illustrate that a nutritional diary describing a patient's nutritional history is stored and can be accessed to display selected information and determine adjustments to nutritional intake.

The displays 300, 370, and 390 can further include an interruption indicator legend 360 that indicates an interruption event that can require compensation in formula administration. The indicator, can indicate (e.g., graphically or textually) the type of event associated with the indicator that was detected. A window with more information about the indicator can be provided, e.g., as an automatic pop-up window or a new window in response to activation of the indicator legend.

When an interruption in formula administration is detected, the interruption indicator legend 360 is activated to visually indicate that an event has been detected. An indicator, e.g., graphical or textual, can indicate that the detected event is a detected interruption in formula administration. In response to the detected interruption, a deficit in administered patient nutrition is determined relative to the patient's nutritional requirements. Additionally, a recommendation can be determined for formula parameters that can compensate for the deficit and be compatible with the patient.

An interruption in formula administration can be detected by monitoring the received measurement data. When the measurement data is received from a device that measures weight of actual formula administered, the measured weight can be converted to volume, and the volume can be monitored over time to determine the actual administration rate. Similarly, when the measurement data is received from a flow meter or captured image data of the display of a pump 102, the measured rate of flow or volume can be converted and/or monitored over time. When the measured weight, rate of flow, or volume of formula administered indicates that no formula was administered, or the amount was decreased, the time period is determined for which no formula or a decreased amount was administered. The measured nutrition delivered over a selectable period of time is compared to the patient's nutritional requirements for that time period, and a nutritional deficit for a selectable nutrient that needs to be compensated for can be determined.

As described in greater detail below with reference to FIG. 7, a recommendation for compensating for the nutritional deficit can be calculated using the patient's present nutritional requirements, predicted nutritional requirements for the patient, and information in nutrition database 118. The patient's predicted nutritional requirements can be determined based on a prediction of the patient's response to the formula to be administered using the data from the patient's nutritional history and medical record, the recommended formula parameters, data indicating a medical procedure planned for a future time, and aggregated data from aggregated knowledge base 122. The recommendation can include one or more compensatory feedings or adjustments in formula parameters. Compensatory feedings can include one or more bolus feedings. Adjustments in formula parameters can include a change in formula and/or a change in administration parameters. The duration for the adjustment can be selected. Additionally, the adjustment can be uniform or change over time, such as by gradually increasing administration rate. Movement indicator legend 362 is activated when a movement event is detected that can affect how the measurement data is processed for calculating the amount of formula administered to the patient. The movement indicator's legend 362 can visually indicate that a movement event has occurred. An indicator, e.g., audial, graphical and/or textual, can indicate that the detected movement event points to a need to modify the calculation of the amount of formula administered to the patient.

Movement events that can affect how the measurement data is processed include change of a formula container; patient transport ambulation or other appropriate movement; and patient thrashing or inappropriate movement. These movement events can affect the measurement data received from the measurement capture device 108 that is used to determine administered formula weight or volume. Thus, substantial changes in measurement data that can be correlated with a movement event can be stored, but set aside and not processed for determining the amount of formula administered to the patient. The measurement data that is set aside can be labeled or tagged as having been associated with a movement event and not processed.

Although not shown, a movement legend and/or an interruption legend can be included with screens 370 and 390 that indicates detection of a movement event or an interruption in formula administration.

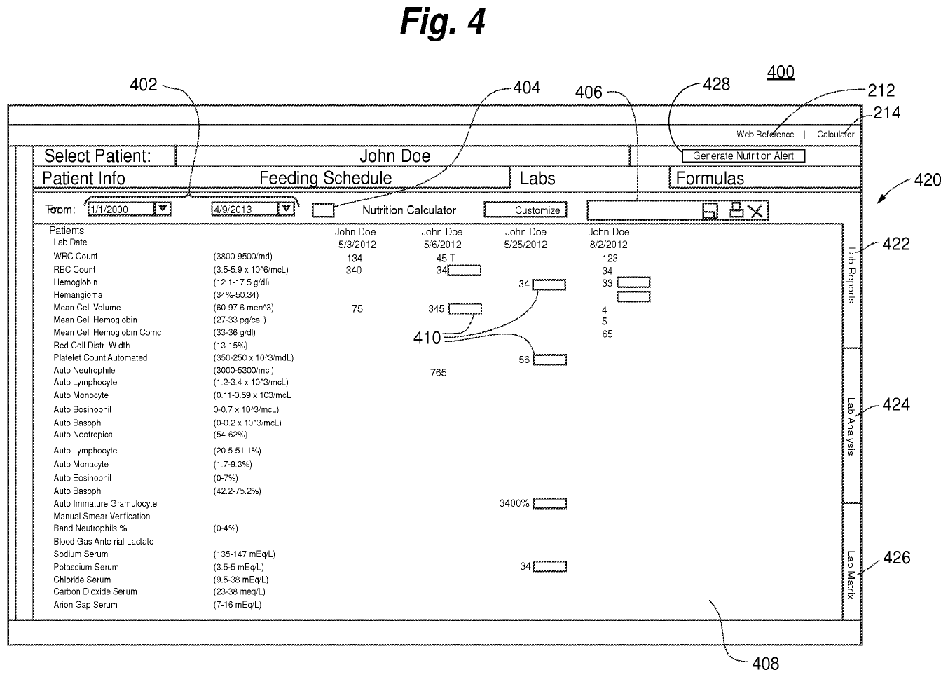

Screen 400 shown in FIG. 4 displays diagnostic results and results from tests performed on the selected patient (e.g., laboratory, radiology, imaging, EEG, and EKG tests). The information can be entered manually or automatically populated from the LIS database 114b. Date/time tab 402 can be used to select a time range for displaying diagnostic result information for the patient. Search tab 404 can be used to search for requested information. Customize tab 406 can be used to select only particular diagnostic results to be displayed. Results window 408 displays diagnostic tests performed, the dates of performance, test results with expected ranges, and an indication (e.g., highlighting with a color) when the test results are high or low relative to the expected range.

The diagnostic results are integrated with the nutrition information. Abnormal diagnostic results can be used to select administration parameters. Diagnostic tests, such as blood tests, that are related to a condition that impacts a patient's nutritional status can be monitored. Such diagnostic tests can include, for example, testing of trending Pre albumin, albumin, electrolytes, vitamin levels, liver function, and kidney function. Trends in abnormal laboratory results for a patient or a plurality of patients can be correlated to nutritional history information. Comparisons can be performed between different patients of abnormal lab results, such as for pre-albumin and other micro/macro nutrition related factors.

Menu 420 along the right margin of the screen 400 provides a selection of display modes. The lab reports display mode tab 422 is currently selected in screen 400. When the lab analysis mode tab 424 is selected, a graph is displayed that shows trends for a selected patient, and can also analyze and display trends for a selection of multiple patients. When the lab matrix mode tab 426 is selected, lab results for patients that have associated data that satisfy a selected condition are displayed in a graphical format. The lab analysis and lab matrix views provide a research tool that a user can use to correlate lab analysis values and patient nutrition history information and identify trends. In an example, a trend was detected for a group of selected patient that had a similar medical condition and underwent similar operative procedures. The patients were administered different enteral nutrition formulas. It was detected, that patients using a particular formula exhibited elevated blood sugars relative to the other patients. Trends can also be determined in relation to the progression of recovery of the patients. Researchers and providers can optimize nutrition support for future patients based on research and evidence based practice.

The ability to detect trends may motivate healthcare providers to pay closer attention to basic serum and blood markers of malnutrition that correlate with nutritional status, such as albumin, pre-albumin, potassium, sodium, and the like. The analyses performed in connection with the lab analysis and lab matrix views can include accessing aggregated patient data in aggregated database 122 and the nutrition database 118. Additionally, the results can be used to update the aggregated database 122 and nutrition database 118 to indicate trends and influence recommendations and predictions, such as those described in connection with screen 300.

Additionally, diagnostic information, such as lab test results and medical diagnostic test results, can be used to determine how and when to compensate for a deficit in nutritional administration. Such test results can be used to determine whether to change the cumulative or incremental surplus and/or deficit thresholds. For example, when the lab test results indicate an abnormal albumin or pre-albumin level, the cumulative threshold or incremental threshold can be adjusted. In an example, the incremental or cumulative threshold for determining when to provide compensation can be adjusted e.g., increased, when lab test results indicate that retention of fluids can be harmful, for example when the lab test results show that the patient has an elevated creatinine level that indicates that that the kidneys may be malfunctioning and not flushing fluids adequately. The degree that the incremental or cumulative threshold is raised can be correlated to the elevation of the indicator of kidney malfunction

In a further example, when lab test results indicate that the patient has elevated albumin or pre-albumin levels that can be indicative of a protein deficit, the incremental or cumulative thresholds can be adjusted, e.g., decreased, to increase the responsiveness of the compensation.

In another example, the patient's medical diagnosis can be used to adjust the incremental or cumulative thresholds. For example, when a patient has renal failure, the incremental or cumulative threshold can be changed to account for a need to reduce fluid volumes. In another example, a diabetic diagnosis can cause adjustment of the incremental or cumulative thresholds for nutrients that affect blood sugars.

Screen 400 can further include a tab 428 (or setting) that can be actuated (or set) to generate an indicator when a diagnostic result is abnormal and may indicate that a nutritional adjustment should be considered. The setting can be set to generate indicators for particular types or degrees of abnormal results. When an abnormal diagnostic result occurs, the user can be queried regarding whether or not the indicator should be generated. The indicator can be displayed on one or more screens associated with the patient's nutritional care. An example indicator window 528 in screen 500 (See FIG. 5) is shown in which an indicator is displayed. The indicator can be generated as an audio indicator. The indicator can be generated by sending an email, SMS text message, or voicemail to a designated medical practitioner.

The indicator displayed in indicator window 528 can indicate what the condition was that caused the alert (e.g., the diagnostic result that was abnormal). Additionally, the indicator can indicate recommendations for nutritional intervention. For example, recommendation to change the amount of protein administered can be recommended when the diagnostic result indicated a change in albumin level.

Thus, diagnostic result data related to the patient's medical condition can be integrated with the nutrition administration information for the patient and the nutritional requirement data for the patient by alerting a medical practitioner that a nutritional adjustment may be needed.

Nutritional input screen 500 shown in FIG. 5 displays nutritional information for a variety of enteral formulas. Although not shown, screen 500 can also include nutritional information for other nutritional intakes, such as supplemental formulas and/or oral nutrition items. The information is updated periodically. The information can be accessed when generating recommendations or calculating predictions, such as described above in connection with screen 300. One or more customize tabs 502 can be used to select which formulas and nutritional information (nutrients, micronutrients, and/or macronutrients) should be displayed and/or which units to use for measured values. For example, a health care institution may only provide a limited choice of enteral formulas or food items, and the provider can select to display only those choices.

The information provided in screen 500 can further include compatibility information about the formulas. Compatibility can include whether the formula is available to the patient (e.g., whether the patient's place of residence has the formula in inventory or can get it). Compatibility can further include whether the patient is allergic to the formula, and whether the patient has the ability to pay for the formula, e.g., based on the cost of the formula and the patient's insurance coverage. Additionally, compatibility can include whether the formula's storage requirements (e.g., temperature requirements) can be met at the patient's residence.

Compare tab 506 can be used to select a standard or a nutritional intake to function as a reference to which to compare the displayed formula(s). In the current example, the selected reference is the RDA standard. Formula results window 508 displays the selected nutritional information results for each formula displayed. In one example, the displayed information for a formula can include the amount of each nutrient provided by the nutritional intake per a selected unit. In another example, the displayed information includes a relative indicator (e.g., a percentage or ratio) that indicates the amount of the nutrient relative to the selected reference in accordance with a comparison between the formula and the selected reference. The displayed result entries 510 can be provided with graphical indicators 512 (e.g., font size, color, or type; highlighting) to indicate when the comparison is above or below a reference value. Since any formula can be selected to be the reference, the formulas can be compared to one another, in any combination. The selected reference formula can be visually indicated, such as highlighted, to identify it as the reference formula to which other formulas are being compared.

In an example, a patient with a low pre-albumin or low albumin level may benefit from a nutritional formula that is higher in protein. A provider can use screen 500 to compare the available enteral formulas and search for the formula with the highest protein. The provider can thus use the functionality of screen 500 to select an appropriate formula in accordance with the patient's medical condition. The nutritional input screen 500 can also display information about the cost or availability of the formulas. Additionally, nutritional input screen 500 can be customized for a patient with reference to cost and availability, including displaying the cost to the patient after insurance information is factored in. For example, if an enteral formula is not available, such as due to an allergy or intolerance of the patient, an inventory deficit or a recall, the information can be displayed in a lighter color or shade (e.g., grayed-out) to indicate that it cannot be selected or operated by the user. Additionally, a comparison of two or more formulas can include a comparison of cost, availability, and insurance coverage for the patient.

Additionally, nutritional input screen 500 can be customized for a patient with reference to nutritional requirements. Tab 516 can be activated by the user to select one or more nutrients, micronutrients, and/or macronutrients. The provider can use the “+” tab 518 to add selections. The patient's nutritional requirement for one or more selected nutrients is displayed in window 520. The user can adjust the requirement using adjustment tab 522. The monitoring system 100 can recommend one or more nutritional intakes that best satisfy the patient's selected nutritional requirements with the highest selected value provided in window 524. The form of nutritional intake (enteral formula “E” or food “F”) available or required for the patient is displayed in window 526. The user can select or deselect a form of nutritional intake, however, the forms that are not available to the patient cannot be selected (e.g., they are grayed out).

With reference to FIG. 6A, a dietician risk screen 600 is shown, which provides access to a patient's nutritional assessment, nutritional risk, nutritional status, nutritional evaluation results, and nutritional history during a selected time period, wherein the nutritional history is based on measurements of administered formula. This screen is beneficial for scrutinizing the nutritional care of patients who have developed a pressure ulcer (e.g., acquired a new pressure ulcer that they did not previously have or that had previously healed). In some jurisdictions, administrative requirements have been instituted that require a nutritional risk evaluation to be performed upon admission of a patient to a health care facility. If the evaluation indicates the existence of a nutritional deficit, a nutritional specialist, such as dietician, can be accessed in order to perform a thorough assessment.

The nutritional status for a selectable time period is displayed in status area 602 to indicate if there is a deficit or surplus associated with the selected time period. Medical status and history information is displayed in medical information area 604. Area 604 can be configured to receive a numerical data entry that indicates the patient's condition and can be analyzed to help determine a condition for which a nutritional adjustment may be recommended. For example, the patient's output (e.g., urine, emesis, stool, diarrhea) can be assessed. A code can be associated with the various outputs. Output occurrences can be entered by user entry and timestamped. The entry can include a numeric volume and a numeric rating for various factors such as color, odor, texture, and patient's overall wellbeing.

The dietician can make a recommendation that is documented in dietician recommendation area 606. These recommendations are readily accessible to the patient's healthcare givers in charge of administering nutrition, including nurses and doctors. Directions for the patient's nutritional care and conformance to the directions can all be recorded and timestamped using the monitor system 100, well documented, and accessible via the dietician risk screen.

In one embodiment, when a patient has a skin wound or skin integrity is lost, the nutritional history is correlated with the quality and rate of the wound healing. An indicator is generated when poor wound healing can be correlated to unwarranted nutrition deficits. Certain conditions may render a nutrition deficit to be warranted, such as organ or multi-organ failure, eating disorder, sustained malnutrition and malabsorption. Additionally, patients with medical conditions coupled with decreased activity level, immobility, and/or incontinence of stool or urine, are at a higher risk of developing skin breakdown and pressure ulcers.

Additionally, if a patient develops a pressure ulcer, the health care facility can risk refusal by insurance companies or government agencies of reimbursement for costs associated with care of the pressure ulcer. The refusal of reimbursement can be based on a presumption that the pressure ulcer developed due to poor nutritional care that did not meet expected standards. However, the health care facility may be able to rebut the presumption of poor nutritional care by showing that proper nutritional care was given, and that poor nutritional care is not a factor that caused the pressure ulcer.

With reference to FIG. 6B, screen 650 displays information related to a skin wound, such as a pressure ulcer. The condition of the wound is recorded. User entry areas 652 and 654 provide information that can be used to identify the wound. The wound type e.g., pressure ulcer stage I-IV, unstageable pressure ulcers, partial thickness wound, full thickness wound, deep tissue injury, skin tears, and incontinence associated dermatitis, is entered and/or viewed in entry area 652; the location of the wound on the patient's body is entered and/or viewed in user entry area 654.

User entry areas 656 and 658 display and/or can accept user entry data that indicates the condition of the wound and whether or not it is healing or getting worse. The size of the wound is entered and/or viewed in user entry area 656. Dimensions of the wound that can be entered as numeric data into user entry area 656 include length (L), width (W), depth (D), undermining (U), and tunneling (T).

At least a portion of the wound dimension data displayed in user entry area 656 can be determined and/or provided by a computing device, such as an imaging device, an imaging processing device, and/or a digital measuring device. For example, an imaging device can capture one or more images of the wound. A person or an imaging processing unit can determine from the image(s) one or more dimensions, and/or the size of the wound. The image processing unit can use information, such as the distance of the imaging device from the wound at the time of wound capture to determine dimensions of the wound. The image processing unit can further analyze the image using image processing techniques, such as edge recognition, to determine wound size. In an embodiment, a gauge can be used to measure one or more dimensions of the wound. In an embodiment, the gauge and the gauge measurement can be captured in the image. In an embodiment, the gauge can be a digital device that includes a processing device and communication device to store and transmit measurement information about the wound.

Images or data from the imaging device, image processing device and/or digital measuring device can be entered and stored using the browse tab 674 and viewed using the uploaded image tab 672.

A rating (e.g., scale 1-10) and/or description of the wound condition and progression, such as relative to the previous assessment, is entered and/or viewed in user entry area 658. The rating can be determined and entered by a human. An image of the wound can be stored, such as to collaborate the wound condition rating. In an embodiment, the wound condition rating can be generated using imaging processing of the image of the wound.

With returned reference to the dietician risk screen 600 of FIG. 6A, access to the initial evaluation; dietician assessment and recommendations; doctor's guidance, documenting each report that a doctor reviewed; instructions generated by the doctor; and/or nutritional history of formula administered to the patient as measured by the measuring capture device 108 are provided to show compliance with recommendations and instructions. Since the nutritional history information is automatically measured and entered, it is not prone to human data entry errors. The nutritional history can show gaps in nutrition administration that were detected and how the detected gaps were compensated for so that the patient received the nutrition that was needed.

Pressure ulcer assessment can be triggered when a patient has developed a pressure ulcer on admission for care at, or by, a healthcare organization (e.g., hospital, home care agency, hospice care agency, rehabilitation facility, long term care facility, etc.) or develops a pressure ulcer while under such care (e.g., a hospital acquired pressure ulcer). The pressure ulcer assessment can be entered and monitored in the nutrition application system. This monitoring allows providers to monitor improvement or worsening of pressure ulcer and alter nutrition or nutrient administration to improve wound healing. Additionally, compliance with nutritional intervention recommendations can be stored, determined, and tracked.

A correlation can be made between the condition of a wound and nutrition care. The correlation can indicate whether compliance with nutritional intervention recommendations contributed to improvement or worsening of the wound. By studying correlation trends among a plurality of patients, lessons can be learned to improve nutrition interventions for promoting prevention of wound development and wound healing.

The information displayed in dietician risk screens 600 and 650 can be useful to store and retrieve documentation that can be used to form a proper rebuttal or demonstrate improper nutrition care. Since the nutrition history includes information about the patient's medical history and condition, a determination can be made whether gaps in nutrition are warranted or not, and whether or not improper compensation for gaps is warranted or not. Additionally, awareness that the development of a wound and its condition are monitored and correlated with the patient's nutrition history can promote an understanding of the effect of nutrition on wound development and healing, and vigilance in nutrition care.

In an example of an intervention, when a wound has developed, increased in size, or the condition has worsened, as indicated by the information that is stored and displayed in screen 650, an indicator can be generated. Screen 650 can further include a tab 676 (or setting) that can be actuated (or set) to generate an indicator when a wound condition has developed or worsened and may indicate that a nutritional monitoring and/or an adjustment should be considered. The setting can be set to automatically generate indicators for particular wound conditions, such as entry of data about a new wound, a change in one or more wound dimensions that exceed a predetermined threshold, and/or the rating of the wound condition has declined by a predetermined amount or to a predetermined level. When the wound condition has been detected, the user can be queried regarding whether or not the indicator should be generated. The indicator can be displayed, e.g., in indicator window 528 in one or more other screens (e.g., screens 500 and 600), generated as an audio indicator, and/or generated by sending an email, SMS text message, or voicemail to a designated medical practitioner. Thus, skin wounds can be monitored and data generated or entered in accordance with the monitoring can be integrated with the nutrition administration information for the patient and the nutritional requirement data for the patient by alerting a medical practitioner that a nutritional adjustment may be needed.

The indicator displayed in indicator window 528 can indicate what the condition was that caused the alert (e.g., medical condition in general, diagnosis, diagnostic result, or wound condition). The indicator can further indicate why the indicator was generated (e.g., the diameter D of the wound exceeded the predetermined threshold set for the patient). Additionally, the indicator can indicate recommendations for nutritional intervention. When the indicator is due to a wound condition, the recommendation can be to administer a bolus of a nutrient that is beneficial for skin healing, such as omega-3 fatty acids, vitamin C, or vitamin E.

Each time that an indicator is generated, a record of the indicator with a timestamp is stored with the patient's nutritional history. This can provide additional documentation to show that the nutritional care of the patient was correlated with the patient's wound care and was vigilant.

With reference to FIG. 7, screen 700 displays formula recommendation information for the patient, including for formula administration parameters. A recommendation can be for a formula or formula parameter due to a change in patient status, or to compensate for a detected or predicted deficit or surplus. The recommended formula can be a new formula or the original formula that was being generated. Time period area 702 displays a selectable accumulation time period for which a deficit or surplus can be determined. The accumulated deficit for the selected time period is displayed at deficit area 704. The selected time period can include future time. Accordingly, the accumulated deficit displayed can include a predicted deficit. Deficit area 704 includes tabs via which a user can select the nutrient for which a deficit is displayed. In the current example, the accumulated deficit displayed is 8220 calories.

Compensation area 706 displays a compensation time period during which the deficit is to be compensated and tabs for selecting the compensation time period and periodic time units (e.g., hours or days). In the example shown, the compensation time period displayed is 37 days.

Periodic portion area 708 displays a portion of the periodic time unit for which compensation is to be provided. When the periodic time unit is selected to be “day,” periodic portion area 708 displays the number of hours out of a 24 hour daily time period that the compensatory amount is administered. In the example shown, the compensatory amount is intended to be provided for 18 hours out of a 24 hour day. Percentage of deficit area 710 displays a percentage of the deficit or surplus that is to be compensated for. The provider may require that an entire deficit (e.g., 100%) be compensated for, or only a portion thereof (e.g., 75%).

Recommendation area 712 displays a list of recommended formulas and formula administration parameters, listed in order of most-to-least recommended. In the example shown, the most recommended formula is Jevity, which is recommended to be administered at an increased amount of 77 cc/hr for 18 hours out a 24 hour day, and 67 cc/hr for the remaining 6 hours of the 24 hour day. Accordingly, the increased amount of 77 cc/hr includes an additional 10 cc/hr relative to the baseline amount of 67 cc/hr. In one embodiment, the medical practitioner selects a formula from the list of recommended formulas. In another embodiment, one of the formulas is automatically selected for administration, and the medical practitioner can override the selection.

The recommendation of one or more formulas can be based on an initial patient status, such as the patient's demographics, medical diagnosis, and medical history. The initial medical history can include, for example, previously stored diagnostic test results, an existing skin wound, a burn condition). The recommendation can also be based on a change in the patient's status, such as an update to the patient's demographics, medical diagnosis, diagnostic test results, skin wound condition, burn condition. For example, a formula with a high concentration of protein may be recommended for a patient that was admitted with a burn. A new formula may be recommended for a patient for whom diagnostic test results indicate a change or imbalance in electrolytes, an abnormal lab result, or a change in wound condition.

When a recommendation is made or implemented, the recommendation itself is stored and timestamped with the patient's nutritional history. The reason for the need to compensate is also recorded and timestamped. For example, the compensation may have been requested by the provider, or the need may have been detected based on a change in the patient's status. When a formula selection or a formula administration parameter includes an adjusted amount due to compensation, the current rate of administration displayed (see FIG. 8) can be displayed with a visual indication (e.g., color, blinking, or highlighting) that it includes compensation. Additionally, the compensation information can be displayed with the nutrition calculations in the patient summary area 350.

The servers 104 or 106 can determine when a change in the patient's status has occurred that would changes the patient's nutritional requirements such that a new or updated recommendation is needed to compensate for an actual or a predicted deficit or surplus.

A change in the patient's status that warrants a recommendation for an updated formula calculation, a new formula, new formula administration parameters, and/or a compensation, can occur, for example, due to a user-entered update to the patient's status parameters or formula parameters; a detected movement event, bag change event, or other interruption event; a change in the patient's records in the HIS database 114a, LIS database 114b, and/or AIS database 114c; a change in other digital patient records accessible by the processor assembly 101; a condition detected by a sensor device 116; development of or a change in the condition of a skin ulcer; or information provided by the pump 102 and/or scanner device 110.

Patient status parameters that can affect the patient's nutritional requirements include, for example, the patient's health status (e.g., a specified disease, gastric condition, heart condition, smoker, etc.); prescribed medication; prescribed medical intervention (e.g., surgery, physical therapy, etc.); weight; diagnostic result (e.g., laboratory, radiographic, or diagnostic results); activity level; feedback (about, e.g., energy level, gastric comfort, etc.); caregiving level available; place of residence (e.g., a move back home, to a hospital, nursing home, intensive care unit, etc.); patient financial means; patient insurance coverage; and patient preference of formula.

Upon detection or observation of an event, such as patient emesis or diarrhea, an analysis can be performed to investigate a possible cause related to administration of formula to the patient. The investigation can include, for example, retrieval and examination of a 48 hour history of formula administration and health status prior to the emesis or diarrhea event.

The recommendation can be determined based on compatibility of the recommended formula with the patient's needs (e.g., considering the formula's allergens, storage requirements, cost, and availability). Additionally, the recommendation can be calculated using the detected change in the patient's present nutritional requirements. The recommendation can include one or more compensatory feedings or adjustments to formula parameters. Compensatory feedings can include one or more bolus feedings. Adjustments to formula parameters can include a change in formula and/or a change in administration parameters. The duration for the adjustment can be selected. Additionally, the adjustment can be uniform or change over time, such as by gradually increasing the administration rate.

A recommendation can be generated for a hypothetical update. In this case, the recommendation can be calculated using user entered hypothetical updates to the patient status, stored data for the patient, and predicted data (e.g., a predicted deficit or surplus). The recommendations responsive to actual or hypothetical updates can be calculated using the patient's present nutritional requirements and/or predicted nutritional requirements for the patient. The patient's predicted nutritional requirements can be determined using a prediction of the patient's nutritional response to the recommended formula parameters, or a past or future medical procedure. The recommendation can use aggregated data for patients having similar patient status parameters from aggregated knowledge base 122. When storing a recommendation related to a hypothetical update of the patient's status, the stored recommendation includes a field or a flag to indicate that it is based on a hypothetical situation. The hypothetical recommendations may not be displayed with the patient's nutritional history unless requested. The recommendation can include one or more compensatory feedings or adjustments to formula parameters. Compensatory feedings can include one or more bolus feedings. As illustrated in FIG. 7, adjustments in formula parameters can include a change in formula and/or a change in administration parameters. The duration for the adjustment can be selected. Additionally, the adjustment can be uniform or change over time, such as by gradually increasing the administration rate. The recommendation can also include administering additional supplemental nutrients not included in the enteral formula, such as adding protein, vitamins, minerals and the like, to the dietary therapy/regimen of the patient.

The success of implementation of any recommendation generated can be monitored to determine whether the administered adjusted amount of the selected formula actually compensated for the determined differences. Determination of success of implementation can include assessing, at a predetermined time period after implementation of the recommendation was commenced, satisfaction of the patient's nutritional requirements and compatibility of the recommended changes with the patient. This determination can include checking diagnostic results in the LIS database 114b; requesting qualitative and/or quantitative feedback from the patient, a medical provider, or the patient's caretaker; and/or determining if the sensor data 116 indicates patient difficulties, such as problems digesting the formula.

FIG. 8 shows screen 800 for display and/or user entry of formula and formula administration information (e.g., enteral or parenteral formula) via continuous administration. Tabs 810 are used for selecting or indicating the type of administration, e.g., continuous (enteral or parenteral), bolus (enteral) or regular (e.g., orally administered nutrition), or any combination thereof.

In one example, tabs 812, 814, 816, and 818 can be operated for controlling formula administration. In another example, screen 800 displays information received from an enteral feeding pump 102 and monitors the pump 102. The screen 800 can display data in real-time, in near real-time in which there is a predetermined delay in between actual activity at the pump and displayed activity in screen 800. In still another example, screen 800 simulates a pump 102 or a flow of formula that uses gravity rather than a pump to administer the formula to the patient, Accordingly, a pump 102 can be positioned at the patient's bedside, and the pump 102 can be monitored via screen 800 displayed on a user device 112 or 120. The user device can be provided at a nurse's station for centralized monitoring. A doctor can monitor a patient using a user device 112 or 120 even from a remote location, such as when at another hospital or an office. A homecare agency can monitor a plurality of patients that are at their respective homes.

The screen 800 further simulates various activities that may occur at the pump or by remote control of the pump. For example, screen 800 can simulate a change of an enteral feeding bag, a change in the formula administration rate, or a change in the formula being administered. Such events can either be sensed or determined by the actual enteral pump 102 and communicated for display on screen 800, or entered by a nurse or caregiver entering data via screen 800. The data can be entered by a first user (e.g., nurse) using a first user device 112 or 120, and viewed by a second user (e.g., doctor) operating a different user device 112 or 120.

Area 820 displays current settings related to the current formula administration session. Tabs 812 and 816 are used to start and end a formula administration session. A formula administration session can correspond to administration of a formula for a particular time interval, such as a calendar day, a nurse's shift (e.g., a night shift or a day shift), or a continual feed until an event occurs, such as an administration parameter setting is changed). Actuation of tab 814 causes formula administration (or simulation thereof) to pause while a formula bag is being changed, or if there is an intentional interruption to the enteral feeding.

Tab 818 and marker 822 are used to change the level of the enteral nutrition residual volume in the event that there were events not captured by the measuring device. It is contemplated that the software system can operate without having a measuring device in place. Once the enteral feeding schedule is started the system calculates how much nutrition is currently being administered, not taking into account any interruptions or holds. For example, if at the end of a shift, the patient should have received 500 cc but the nurse, or care giver notes that the current residual volume is 900 cc, e.g., due to an interruption of feeding during the day the user can input data to reflect the actual residual amount. The system can then recalculate and show the actual intake of the user. Accordingly, the current system is design to work independent from a measuring device. This can accommodate for monitoring nutrition intake for facilities that do not use enteral feeding pumps for administration of enteral nutrition and use a feeding bag that uses gravity to administer the formula.

Area 820 displays the current status of the current formula administration session for the selected nutrition type. The status includes the date and start that administration was started, the formula used, the goal rate (shown per hour) and the current rate (shown per hour). The goal rate is the administration rate that would satisfy the patient's nutritional requirements without adjusting for any deficits or surpluses. The current rate is the rate the formula is currently being administered yet, which is the goal rate adjusted to compensate for a deficit or surplus. The adjustment can be for a cumulative deficit or surplus, or a deficit or surplus associated with a selected time interval.

A visual indication (such as highlighting or blinking) can be provided in association with the formula or formula administration parameters that were changed. The visual indication can further indicate whether the change was entered by a user or was adjusted based on a recommendation. Area 840 displays the feed history for the current formula administration session, including the history for time sub-intervals (e.g., hourly intervals) of the session. The formula, goal rate, actual rate, start and stop date/time are shown for each administration session.

The feed history can indicate a reason for a feeding session termination, when the feeding session included compensation, and when the patient did not tolerate the feeding session. The information in feed history area 840 can be entered automatically or manually by a user. For example, an ID for the patient, caregiver, formula container, and/or feeding pump can be entered for each feeding session, such as by scanning associated barcode or RFID tags using scanner device 110, or by manual entry.

Administration status of a selected nutrient is displayed at entry fields 842 and 844. In the current example shown, the selected nutrient is calories. The amount of the nutrient given cumulatively for the current day until the current point in time is shown at entry 842. The total target amount of the nutrient, e.g., calories, and/or the total target volume of formula, for the current day is shown at entry 844. Accordingly, the entries in fields 842 and 844 can be used to determine a difference between the target goal and the actual (or simulated) amount of formula that was administered.

Area 350 shows the doctor's orders that were entered for the patient, wherein the nutrition calculations shown in area 350 incorporate the doctor's orders. If needed, the order can be changed, e.g., by a nurse. For example, a verbal or telephone order can be given to the nurse. The order can be carried out prior to the prescribed order being entered into the system, e.g., by the ordering physician. The nurse can enter a comment explaining the reason for the difference between the order currently in the system and the order carried out by the nurse, and the subsequent entry of the change to the order. A historical record of the nurse's actions, the change to the order, and the user that entered the information is stored with the patient's nutritional history information.

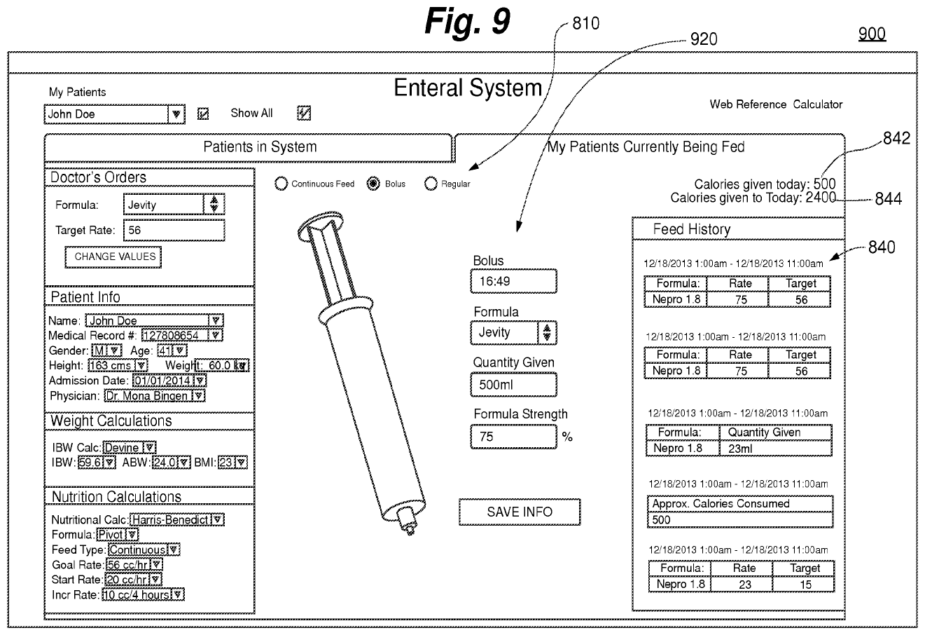

FIG. 9 shows screen 900 for display, simulation, and/or user entry of formula (e.g., enteral) administration via a bolus feeding. Screen 900 can simulate a pump 102 or a flow of formula that uses gravity rather than a pump to administer the formula to the patient as a bolus feeding. The bolus feeding can be used for enteral nutrition feeding and relates to a different option of administering enteral formula over a given period of scheduled feeding rather than continuous over 24 hours via bolus administration. The bolus can include an enteral formula bolus and/or a fluid water bolus that is given for hydration.

Area 920 displays current settings related to the current formula administration session for the selected nutrition type. The settings include the date and time that the bolus was administered, the formula (or water) used, the strength used and the volume administered. Formulas can be diluted and/or are prepared at different strengths. As in screen 800, area 840 displays the feed history for the current formula administration session. The bolus is included as part of the feed history shown, and can be added to the feed history after its administration. As in screen 800, area 830 shows the doctor's orders for nutritional administration.

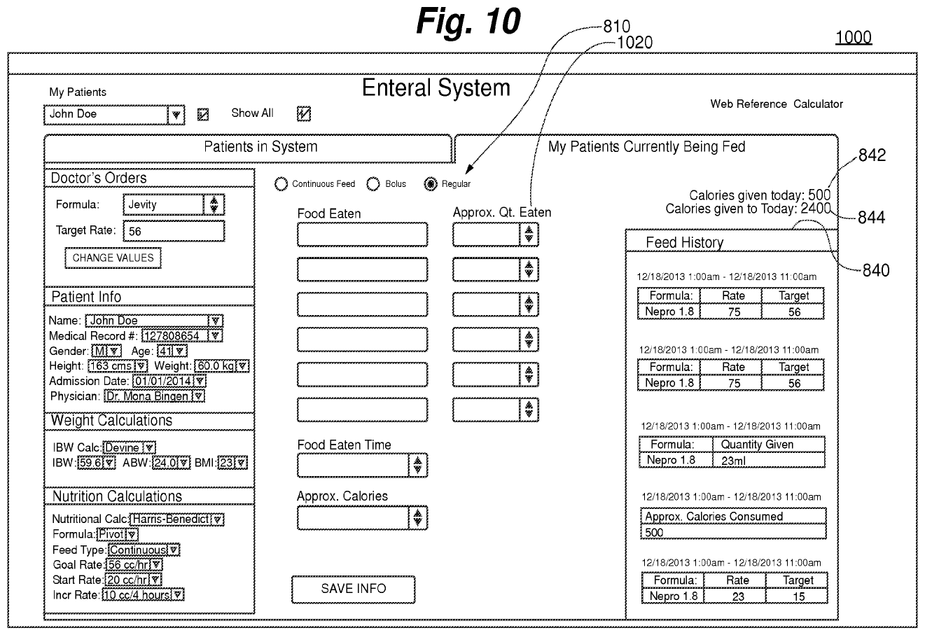

FIG. 10 shows screen 1000 for display, simulation, and/or user entry of supplemental formula or food intake via oral nutrition administration. The monitor system 100 monitors the orally administered nutrition using screen 1000. The orally administered nutrition can be the sole nutrition administered, or can be monitored in in combination with administered parenteral or enteral formula. Area 1020 displays entries for food that was administered orally (eaten, swallowed, and/or drunk by the patient). The system can track the nutritional intake (including calories) of the patient. The settings include fields for entering the date and time for each food eaten by the patient, the name of the food, the approximate quantity eaten, and the approximate nutrient level for a selected nutrient (shown as calories in screen 1000). As in screen 800, area 840 displays the feed history for the current formula administration session. The administration of oral nutrition is included as part of the feed history shown, and can be added to the feed history after its administration. As in screen 800, area 830 shows a general status of the patient and nutrition administration settings.

Feed history area 840 shows that on a particular day, the patient was administered enteral formula and consumed oral nutrition. The nutrients received from both the formula and the oral nutrition are used in the calculations to determine a total amount of a nutrient administered to the patient whether or not there was a deficit or surplus for that day, or cumulatively over a selected time period, relative to the patient's nutritional requirements.

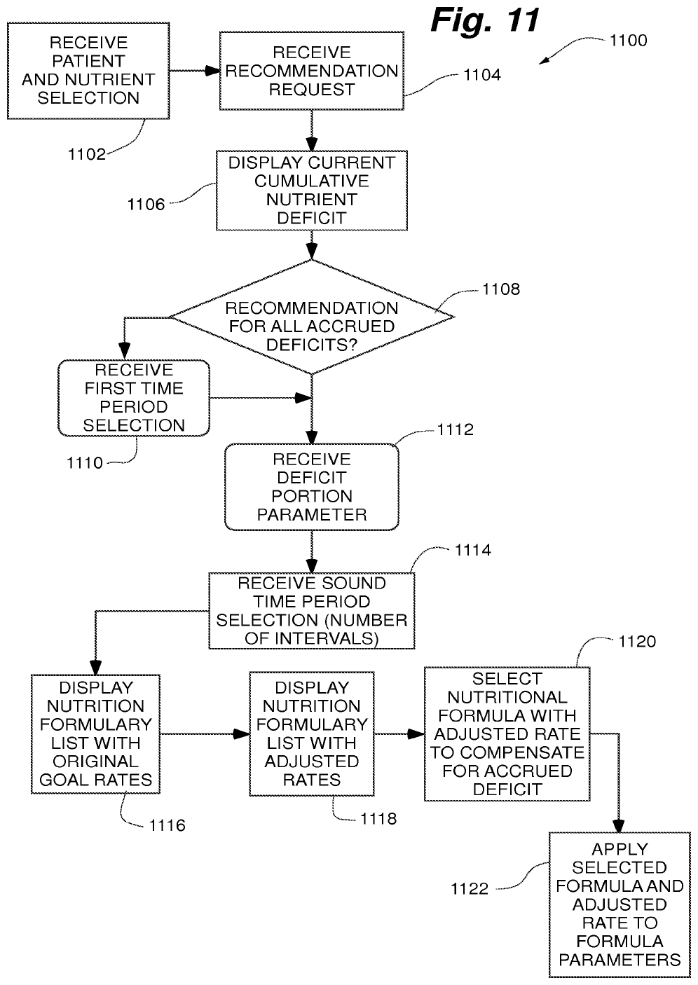

FIG. 11 shows a flowchart 1100 that illustrates an example method of the monitor system 100 shown in FIG. 1. Input that is received by the method, such as requests or selections, can be received by user input or from another process that is executing. At step 1102, a patient selection and nutrient selection (e.g., calories or protein) are received. At step 1104, a recommendation request is received. The recommendation request can be received when a user activates the nutrition calculator button 214 shown on the screens shown in FIGS. 2-10. At step 1106, the current cumulative deficit for the selected nutrient (e.g., calories or protein) is displayed.

At step 1108, a determination is made whether the recommendation request is for all accrued deficits. The determination can be made by querying the user or the requesting process, or determining if a time period selection is received for a first time period that the recommendation is being requested for. If it is determined that the user is not requesting a recommendation for all accrued deficits, but is requesting a recommendation for selected first time period, then at step 1110 a selection of the first time period is received that specifies the beginning and end of the first time period, after which the method continues at step 1112. If it is determined that the user is requesting a recommendation for the all accrued deficits for the selected patient, then the method continues at step 1112.

The time period for which the recommendation is to operate on, which either the first time period or another default time period (e.g., such as since the first deficit or surplus was detected, since nutrition administration via enteral or parenteral administration was commenced, since monitoring of nutrition administration commenced, or another default time period, such as two months. The time period includes intervals. For example, the time period can be measured in units, such as hours or days. The time period has subintervals, such as minutes or hours.

In an example, the time period is 37 days, having 37 day intervals, and each day interval having 24 hour subintervals. In another example, the time period is 36 hours, having 36 hour intervals, and each hour interval having 60 minute subintervals.

At step 1112, a deficit portion parameter is received. The deficit portion parameter specifies a portion of sub-intervals for each interval that an adjustment can be made for. At step 1114, a second time period selection, which can be described as a selected number of intervals is received. The second time period specifies the duration over which an adjustment (if needed) to administration of the selected nutrient is to occur.

In an example, the second time period is selected to be 37 days and the deficit portion parameter received is 18/24 hours. Accordingly, an adjustment to compensate for the deficit is calculated to be administered for 37 days for 18 hours of each day.

At step 1116, a list of one or more formulas (e.g., enteral or parenteral) is displayed with an original goal rate displayed for each listed formula. The original goal rate is the rate that the formula would be administered at if a deficit had not been accrued. At step 1118, an adjusted rate is determined and displayed for each of the listed formulas. The adjusted rate is calculated to compensate for the nutritional deficit of the selected nutrient that the recommendation is selected for, in accordance with the selected second time period and deficit portion parameter. At step 1120, a selection is received for one of the listed formulas. At step 1122, the selected formula and its adjusted rate are applied to the formula parameter and formula administration parameters for the second time period in accordance with the deficit portion parameter.

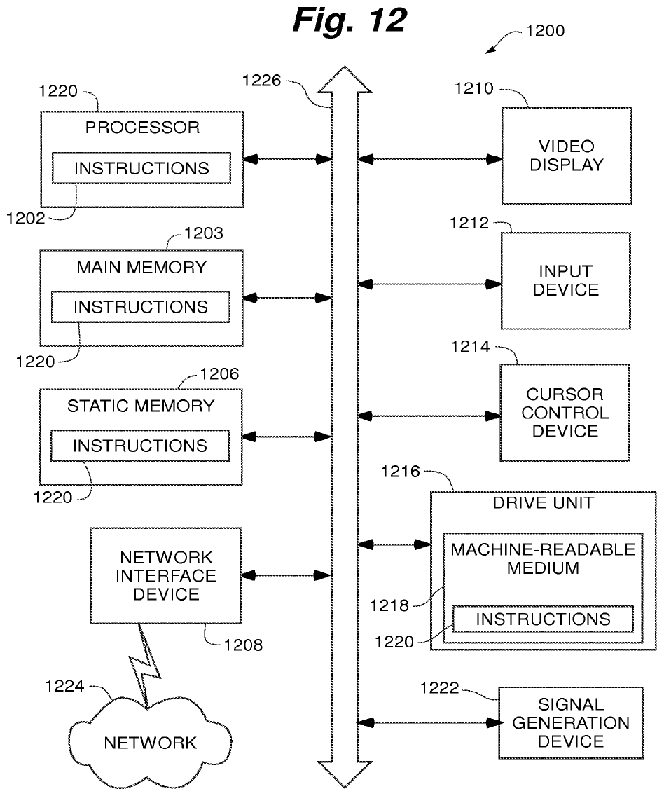

FIG. 12 is a block diagram of an illustrative embodiment of a general computer system 1000. The computer system 1200 can be the local user device 112, global user device 120, enteral facility server 104, and the enteral web server 106 illustrated in FIG. 1. The computer system 1200 can include a set of instructions that can be executed to cause the computer system 1200 to perform any one or more of the methods or computer based functions disclosed herein. The computer system 1200, or any portion thereof, may operate as a standalone device or may be connected, e.g., using a network or other connection, to other computer systems or peripheral devices. For example, the computer system 1200 may be operatively connected to a different user device 112 or 120, a different server 104 or 106, a different processor of scanner device 110, feeding pump 102, parenteral formula pump 124 (e.g., an infusion pump), or databases 114a-114c, 118, and 120.

The computer system 1200 may also be implemented as or incorporated into various devices, such as a personal computer (PC), a tablet PC, a personal digital assistant (PDA), a mobile device, a palmtop computer, a laptop computer, a desktop computer, a communications device, a control system, a web appliance, or any other machine capable of executing a set of instructions (sequentially or otherwise) that specify actions to be taken by that machine. Further, while a single computer system 1200 is illustrated, the term “system” shall also be taken to include any collection of systems or sub-systems that individually or jointly execute a set, or multiple sets, of instructions to perform one or more computer functions.

As illustrated in FIG. 12, the computer system 1200 may include a processor 1202, e.g., a central processing unit (CPU), a graphics-processing unit (GPU), or both. Moreover, the computer system 1200 may include a main memory 1204 and a static memory 1206 that can communicate with each other via a bus 1226. As shown, the computer system 1200 may further include a video display unit 1210, such as a liquid crystal display (LCD), an organic light emitting diode (OLED), a flat panel display, a solid state display, or a cathode ray tube (CRT). Additionally, the computer system 1200 may include an input device 1212, such as a keyboard, and a cursor control device 1214, such as a mouse. The computer system 1200 can also include a disk drive unit 1216, a signal generation device 1222, such as a speaker or remote control, and a network interface device 1208.

In a particular embodiment or aspect, as depicted in FIG. 12, the disk drive unit 1216 may include a computer-readable medium 1218 in which one or more sets of instructions 1220, e.g., software, can be embedded. Further, the instructions 1220 may embody one or more of the methods or logic as described herein. In a particular embodiment or aspect, the instructions 1220 may reside completely, or at least partially, within the main memory 1204, the static memory 1206, and/or within the processor 1202 during execution by the computer system 1200. The main memory 1204 and the processor 1202 also may include computer-readable media.

In an alternative embodiment or aspect, dedicated hardware implementations, such as application specific integrated circuits, programmable logic arrays and other hardware devices, can be constructed to implement one or more of the methods described herein. Applications that may include the apparatus and systems of various embodiments or aspects can broadly include a variety of electronic and computer systems. One or more embodiments or aspects described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

In accordance with various embodiments or aspects, the methods described herein may be implemented by software programs tangibly embodied in a processor-readable medium and may be executed by a processor. Further, in an exemplary, non-limited embodiment or aspect, implementations can include distributed processing, component/object distributed processing, and parallel processing. Alternatively, virtual computer system processing can be constructed to implement one or more of the methods or functionality as described herein.

It is also contemplated that a computer-readable medium includes instructions 1220 or receives and executes instructions 1220 responsive to a propagated signal; so that a device connected to a network 1224 can communicate voice, video or data over the network 1224. Further, the instructions 1220 may be transmitted or received over the network 1224 via the network interface device 1208.

While the computer-readable medium is shown to be a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the methods or operations disclosed herein.

In a particular non-limiting, example embodiment or aspect, the computer-readable medium can include a solid-state memory, such as a memory card or other package, which houses one or more non-volatile read-only memories. Further, the computer-readable medium can be a random access memory or other volatile re-writable memory. Additionally, the computer-readable medium can include a magneto-optical or optical medium, such as a disk or tapes or other storage device to capture carrier wave signals, such as a signal communicated over a transmission medium. A digital file attachment to an e-mail or other self-contained information archive or set of archives may be considered a distribution medium that is equivalent to a tangible storage medium. Accordingly, any one or more of a computer-readable medium or a distribution medium and other equivalents and successor media, in which data or instructions may be stored, are included herein.

In accordance with various embodiments or aspects, the methods described herein may be implemented as one or more software programs running on a computer processor. Dedicated hardware implementations including, but not limited to, application specific integrated circuits, programmable logic arrays, and other hardware devices can likewise be constructed to implement the methods described herein. Furthermore, alternative software implementations including, but not limited to, distributed processing or component/object distributed processing, parallel processing, or virtual machine processing can also be constructed to implement the methods described herein.

It should also be noted that software that implements the disclosed methods may optionally be stored on a tangible storage medium, such as: a magnetic medium, such as a disk or tape; a magneto-optical or optical medium, such as a disk; or a solid state medium, such as a memory card or other package that houses one or more read-only (non-volatile) memories, random access memories, or other re-writable (volatile) memories. The software may also utilize a signal containing computer instructions. A digital file attachment to e-mail or other self-contained information archive or set of archives is considered a distribution medium equivalent to a tangible storage medium. Accordingly, a tangible storage medium or distribution medium as listed herein and other equivalents and successor media, in which the software implementations herein may be stored, are included herein.

Thus, system and method to reconstruct cardiac activation information have been described. Although specific example embodiments or aspects have been described, it will be evident that various modifications and changes may be made to these embodiments or aspects without departing from the broader scope of the invention. Accordingly, the specification and drawings are to be regarded in an illustrative rather than a restrictive sense. The accompanying drawings that form a part hereof, show by way of illustration, and not of limitation, specific embodiments or aspects in which the subject matter may be practiced. The embodiments or aspects illustrated are described in sufficient detail to enable those skilled in the art to practice the teachings disclosed herein. Other embodiments or aspects may be utilized and derived therefrom, such that structural and logical substitutions and changes may be made without departing from the scope of this disclosure. This Detailed Description, therefore, is not to be taken in a limiting sense, and the scope of various embodiments or aspects is defined only by the appended claims, along with the full range of equivalents to which such claims are entitled.

Such embodiments or aspects of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments or aspects have been illustrated and described herein, it should be appreciated that any arrangement calculated to achieve the same purpose may be substituted for the specific embodiments or aspects shown. This disclosure is intended to cover any and all adaptations or variations of various embodiments or aspects. Combinations of the above embodiments or aspects, and other embodiments or aspects not specifically described herein, will be apparent to those of skill in the art upon reviewing the above description.

In the foregoing description of the embodiments or aspects, various features are grouped together in a single embodiment for the purpose of streamlining the disclosure. This method of disclosure is not to be interpreted as reflecting that the claimed embodiments or aspects have more features than are expressly recited in each claim. Rather, as the following claims reflect, inventive subject matter lies in less than all features of a single disclosed embodiment or aspect. Thus the following claims are hereby incorporated into the Detailed Description, with each claim standing on its own as a separate example embodiment or aspect. It is contemplated that various embodiments or aspects described herein can be combined or grouped in different combinations that are not expressly noted in the Detailed Description. Moreover, it is further contemplated that claims covering such different combinations can similarly stand on their own as separate example embodiments or aspects, which can be incorporated into the Detailed Description.

1. A nutrition monitoring system for monitoring a patient's nutrition administration, the system comprising:
a processing device; and
a memory to store instructions that, when executed by the processing device, cause the processing device to perform operations comprising:
accessing nutrition administration information for a plurality of patients describing nutrition administered by different forms to the plurality of patients;
accessing medical information about the plurality of patients from a healthcare information system;
accessing nutritional requirement data describing at least one nutritional requirement for the plurality of patients;
determining a difference between at least one nutritional requirement for a patient of the plurality of patients based on the nutritional requirement data, an amount of a nutrient administered to the patient, and the nutrition administration information; and
integrating the nutrition administration information for the patient, the medical information for the patient, and the nutritional requirement data for the patient, for determining a nutritional adjustment for administering the amount of the nutrient to the patient based on the difference.

2. The nutrition monitoring system according to claim 1, wherein the medical information includes at least one of a lab result, a diagnostic testing result, a medical diagnosis, a medical condition, and a wound monitoring result for the plurality of patients.

3. The nutrition monitoring system according to claim 1, wherein the operations further comprise:
storing nutrition administration information for nutrition administered to the patient over time;
storing the patient's nutritional requirements over time; and
determining over a selectable accumulation time period an accumulation of the difference for the nutrient,
wherein determining the nutritional adjustment is further based on a percentage of the accumulation of the difference specified by a selectable compensation percentage, wherein the compensation percentage is at least one of received by user input and determined using the medical information.

4. The nutrition monitoring system according to claim 3,
wherein the nutritional adjustment is determined for a selectable compensation time period having a plurality of intervals that each include a plurality of sub-intervals, and for a selectable portion of the sub-intervals for each interval, and
wherein the second time period and the portion of the sub-intervals are at least one of received by user input and determined based on the medical information.

5. The nutrition monitoring system according to claim 1,
wherein the different forms of nutrition administered to the plurality of patients include at least two of enteral formula, parenteral formula, and orally administered nutrition, and
wherein accessing nutrition administration information for the plurality of patients includes receiving measurement data captured from at least one enteral feeding pump or parenteral infusion pump that administers nutrition.

6. The nutrition monitoring system according to claim 1, wherein the operations further comprise generating control signals to control a feeding pump or infusion pump to administer nutrition to the patient based on the adjusted amount of nutrition to be administered to the patient.

7. The nutrition monitoring system according to claim 1, wherein the operations further comprise:
monitoring nutrition status related data associated with the medical information of a patient of the plurality of patients that indicates a change in, or a risk factor associated with, a nutritional requirement of the patient, the nutrition status data related to at least one of development of a skin wound, condition of a skin wound, diagnostic result data, and a medical diagnosis;
generating an indicator when the nutrition status related data indicates at least one of: the patient was admitted to a health care facility with a skin wound, the patient developed a skin wound, the skin wound has increased in a dimension or worsened in condition, a new medical condition has developed, a medical condition has worsened;
recording the indicator and a corresponding timestamp; and
receiving a nutritional intervention recommended by a medical practitioner.

8. The nutrition monitoring system according to claim 7,
wherein the nutritional adjustment is further determined based on the nutrition status related data, and
wherein generating the indicator includes generating a recommendation for determining the nutritional adjustment.

9. The nutrition monitoring system according to claim 1, wherein the operations further comprise:
monitoring nutrition status related data associated with the medical information of a patient of the plurality of patients that indicates a change in, or a risk factor associated with, a nutritional requirement of the patient, the nutrition status data related to at least one of development of a skin wound, condition of a skin wound, diagnostic result data, and a medical diagnosis;
selecting, based on the nutrition status related data, at least one of an enteral formula from a plurality of enteral formulas to administer to the patient, and an administration parameter for administering the selected enteral formula to the patient, the administration parameters including at least one of rate of formula administration, formula volume to be administered, and time duration of formula administration.

10. The nutrition monitoring system according to claim 1, wherein the operations further comprise:
receiving a user input designating one formula of a plurality of formulas as a reference formula;
comparing another formula of the plurality of formulas to the reference formula; and
determining a difference in nutrition content for at least one nutrient between the other formula and the selected formula, wherein
the nutritional adjustment is determined for the reference formula and for the other formula.

11. The nutrition monitoring system according to claim 1,
wherein accessing nutrition administration information for the plurality of patients includes receiving a weight measurement of a reservoir of formula, and
the operations further comprise determining a weight of formula that was actually output by a pump administering the formula from the reservoir based on a change in the received weight measurement.

12. The nutrition monitoring system according to claim 11, wherein the operations further comprise:
receiving expected pump output data describing an amount of formula that the pump is expected to output;
determining a difference between a weight of the amount of formula described by the expected feeding pump output data and the determined weight; and
generating control signals to control the feeding pump to adjust the weight of the formula output by the feeding pump based on the determined difference.

13. The nutrition monitoring system according to claim 11, wherein the operations further comprise determining whether the weight measurement indicates the occurrence of an event related to movement of the bag or patient.

14. The nutrition monitoring system according to claim 1, wherein the nutrition administration describes nutrition of at least two different forms of nutrition administered to the patient, the two different forms selected from enteral formula, parenteral formula, and oral nutrition.

15. The nutrition monitoring system according to claim 1, wherein accessing the nutrition information for a patient of the plurality of patients includes simulating administration of enteral formula by a pump.

16. The system according to claim 15, wherein the pump operates using gravity to administer the formula.

17. The nutrition monitoring system according to claim 15, wherein the pump is remote from the processing device.

18. A processing system for monitoring a patient's enteral feeding, the processing system comprising:
a processing device; and
a memory to store instructions that, when executed by the processing device, cause the processing device to perform operations comprising:
providing a graphical user interface (GUI) configured for a user to:
enter and update a plurality of patient status parameters related to at least one nutritional requirement; and
select at least one nutritional formula from a plurality of nutritional formulas;
receiving initialization information to initialize at least a portion of the plurality of patient status parameters and the selected at least one nutritional formula;
receiving, via the GUI, patient status update information each time a user updates the plurality of patient status parameters;
receiving, via the GUI, nutritional formula selection information each time a user updates the selected at least one nutritional formula;
accessing information describing nutritional content of the plurality of nutritional formulas;
calculating a change in the at least one nutritional requirement of the patient based on received patient status update information;
indicating a nutritional formula of the plurality of nutritional formulas that can satisfy the patient's at least one nutritional requirement based on the change in the patient's at least one nutritional requirement and the nutritional content of the plurality of nutritional formulas; and
calculating a difference between the patient's at least one nutritional requirement and corresponding nutritional content that can be provided by the selected or indicated nutritional formula over a selectable time interval based on received nutritional formula selection information and the plurality of patient status parameters.

19. The processing system according to claim 18, wherein selecting the nutritional formula is further based on at least one of ingredients, cost, and availability of the plurality of nutritional formulas.

20. The processing system according to claim 18, wherein the operations further comprise:
calculating, based on the change in the patient's at least one nutritional requirement and the nutritional content of the plurality of nutritional formulas, formula administration parameters for administering the selected or indicated nutritional formula; and
calculating, based on received formula selection information, the nutritional content of the selected or indicated at least one nutritional formula, and the plurality of patient status parameters, formula administration parameters for administering the selected or indicated nutritional formula,
wherein the formula administration parameters include at least one of rate of formula administration, formula volume to be administered, and time duration of formula administration.

21-31. (canceled)
